SEQUENCE LISTING (20 sequences)

SEQ ID NO: 1 - protein, 599 aa, Saccharomyces cerevisiae
MVSAPESIMKNVENIHSSRLTNVKSVLSATELSIIRSNANLEKPSVPSGCYGRILRKLEVPHDGKPISILRNPDLEPIKLRERKWGFWSFFAYWALPNCSIGTLSTGSALLALNLNVKESIGVLVVSNIIVSLFTIACSNPGIKYHIGYTLDQRLLFGIYGSYLTILIRVGLSIVLYAYLSWMGGLCVNMVFNSFSVHYLNMKNIFPDSVPFVTKDFVGFLCFQLIQMPFSFVRPSLVNVPSIVASLMSLAAVVGMFAYLLTTNSGPGPLYNVKIEMSTKERAWAWIFGITIWYSGVAAPVSNQSDYSRFATGGPSSYWGLSLGSILLGVFVPVSGLICASACKQLYGQAYWSPDQIVTQWLNDSYSAKSRAAAFFIGISFTGSQLFFNLTQNGYSCGMDLAGILPKYINVTRGTLFVQLISWLVQPWTFFNSSSAFLNAVSSFGIFTTPIVAINAVEFFYFRRSTIPLIDFFTLSKEGTYWYTSGFNWKSILSLLAGISLGIPGLVYQVNTGSKINTGMQNFYYGYIFFSPLVSGGLYLILTHLFPVRHEKMCKGDPVDFFNCFNDQERQKMGMLPCGAESGGIYEYLDGEECEDTIE

SEQ ID NO: 2 - DNA, 1799 bp, Saccharomyces cerevisiae
tgctcaatggtatcttcacactcctcaccgtctaaatactcataaatgccacctgattctgcaccgcatggaagcatacccattttttgacgttcctgatcgttgaagcagttgaaaaaatctacagggtcccccttgcacatcttttcatgacgaacaggaaacaagtgtgtcagtattaagtaaagccctccggagacaagcggtgaaaaaaaaatataaccataatagaaattctgcatgccagtgttaatctttgaacctgtgttcacttggtacactaatcctggtatacccagagatattcctgccagtaaactgagaatagacttccaattaaaaccagatgtgtaccagtaagtgccctccttggataaggtaaaaaaatctatcagcggtatcgtactcctcctgaaataaaaaaattcgacagcattaatcgctactatgggtgttgtgaatataccaaacgagcttactgcgtttaaaaatgccgaagatgaattgaaaaaagtccatggttggactagccaggatatcaattggacaaaaagtgtccctcgagtaacattaatatattttggtaatattccagccaaatccatgccgcaggagtatccgttttgggtcaagttaaaaaacagttgagatcccgtaaagcttatcccgataaaaaatgctgctgctcttgacttggcagaatagctatcgttaagccattgagtgacaatttggtcaggtgaccagtatgcttgaccgtataattgcttgcatgctgaagcacatattaatccagagacgggtacaaaaacgcctagcaaaatagaaccgagggaaagtccccaataagaagaggggcctcctgttgcgaaacgcgaataatcagactggtttgaaaccggcgcagcaacaccgctataccaaatagttataccaaagatccaagcccaggccctctctttcgtagacatttctatctttacattgtatagcggacctggccctgaatttgtcgtcagaagataagcaaacataccaaccactgcagccaaagacatcagggacgcaacaatggaaggaacgttaaccaagctgggtctgacaaaagaaaaaggcatctgaatgagttggaaacataaaaatccaacaaaatctttggttacaaacggtacagaatccggaaagatgtttttcatgttcagatagtggacggaaaatgaattgaacaccatgttgacacataagcctcccatccatgagaggtatgcataaagaactatagacagtcccacacgtatcagtatggtgagataggaaccgtaaattccaaatagtagcctttgatcgagagtgtatcctatatgatactttattcctggattagaacaagcaatcgtaaaaagagaaactatgatattagacacaactagaactccaatcgactctttgacatttaggttcagggccagcaatgctgaacctgtcgataatgtacctatggaacagttgggtaacgcccaataggcaaaaaaagaccagaatccccacttacgttcgcgtagctttatgggttccaagtccggattcctcagtatagagatgggctttccatcatgtggcacttccagcttccgtaaaatcctaccgtaacaaccagaggggacactaggcttctccaaatttgcattactcctgattatgctcaattctgttgcactcagaaccgacttcacatttgtcagcctgctgctgtggatattttcaacgttcttcattatactttctggcgctgaaaccat

SEQ ID NO: 3 - protein, 355 aa, Saccharomyces cerevisiae
MIDKMETADPKTSETIKNPNLDWKNHTEQDIETGTTVDTLLVTELVEPTSFISSKWKLYLVYCIVYLCATMQGYDACLMSSLYTMDEYSTYYKLEANSAANASIVFAIYSIGQICASPFIPIMDWLGRRKVIWLGCGLVCIGALVTAVSRDFHTLIGGRWLLSFFTTLVCSAAPAYCVEMAPSKIRGRMTGFYMTLFPLGAFTAAFVSYGTGKGFSGQSNAFKIPLWVQLVFPGIVFLTGWYIPESPRWLVGVGREDEAKAILSNYHFASNTEDPRIDDEILDMKNSFGGKRLSDPLTMLDMRPLFSSRSQIYRFGLVVAIAMIGQCSGNNVMAFFLPTMLYESGIKSASGRVLL

SEQ ID NO: 4 - DNA, 1065 bp, Saccharomyces cerevisiae
atgatcgataaaatggaaactgccgaccctaaaacttctgaaactataaaaaacccaaatttagattggaaaaatcacacggaacaggacattgagactggtacgacagtagatactttgttggtgacggaattagtcgaaccaacttcctttatttcttcaaaatggaagttatatttggtttactgcatcgtttatctttgtgctacaatgcaagggtatgatgcctgtctcatgtcctctttgtacacgatggatgaatattcgacatactataaattagaagctaattctgctgccaacgccagtattgtctttgccatatacagtattggacagatatgtgcctctccatttattccgataatggattggctgggtaggagaaaagtaatatggcttggttgtggtcttgtttgcataggggccttagtgacagctgtaagcagggattttcacaccttgattggtggtcgatggctcctttcctttttcacaactttggtgtgctctgctgctccagcatattgtgttgaaatggctccatcaaagataaggggacgaatgaccggtttctacatgacacttttccctttaggggctttcacagcggcgtttgtgtcttacggaacaggaaaagggttttctggacaaagtaatgcttttaaaatacctctttgggtccagttggtatttccaggaattgttttcttgaccgggtggtatattccggaatcacctagatggttagttggtgttgggcgtgaggatgaagctaaagcaattctttctaactatcactttgcctccaatacggaagatcctagaatagatgatgagatattggacatgaagaactcgtttggtggcaagagactctctgatccgttgactatgcttgatatgagaccacttttcagtagtaggtcgcagatttatcgctttgggctcgtagtagctattgctatgataggacaatgttcaggaaataacgttatggcatttttcttgccaacaatgttgtacgaatcgggcattaaatctgcttccggaagagtgttgtta

SEQ ID NO: 5 - protein, 325 aa, Saccharomyces cerevisiae
MHPYIYCTHISIQLPRSSESNVSSQELDVFRDTICFSDLQFRILQDYYSVEFSRCASLNGPDSEKKAHVERLLQLSAGEQLMEEWWKNVSSKSRFQNNKSFSAAHLQIYILTYKILMNKPLLIHPVQCTTEDICDDLPISVCTSAAKEILDICSKYNLNESLMLPQLIYGIYLSSIIFLFNRYSSNISARNEGDRSFSNGLALLEKHTKARKSVNIYYCNLMMFEKHYKNSFQLSTNSDQIVENENYSQYGSSAQSSHSSVNEFNKVSMPTIAQSLDEPNSVFDPLWSDFSNFLGPLSMADENDDYLANLEESISEKSLQNVVWE

SEQ ID NO: 6 - DNA, 978 bp, Saccharomyces cerevisiae
atgcatccctacatctattgtactcatatttctatacagttaccacggagttctgaaagcaatgtatcctctcaggagcttgacgtattcagggacactatttgcttttctgatcttcagttcagaattttacaagattactattctgttgaattttcaaggtgtgccagtttaaatggacctgacagtgaaaaaaaagcccatgtagaacgtcttttgcaattgtcagcgggagagcagctgatggaggaatggtggaagaatgtaagttcaaagtcaaggtttcagaataataaatctttcagcgcagcccatttacaaatatatatcctcacctataaaattttaatgaacaaaccgttattaatccatccagttcaatgtactacggaagatatttgtgatgacttaccaatttctgtttgtacttctgccgcaaaggaaatactcgatatatgctcaaagtacaacttgaatgagtctctcatgttaccacaacttatctatggtatatacttatcttctattatatttctcttcaaccgctattcttcgaatatctcagcgagaaacgaaggggaccgatcgttttcaaacggcttggctcttttagaaaagcatacaaaggcaagaaagtcagtaaacatctattattgcaatttaatgatgtttgaaaaacattataagaattctttccaactgtctacaaatagtgaccaaattgtcgaaaatgagaattattcacagtatggctcgtctgcccaatcaagtcattcatctgtaaatgagtttaataaagtttcaatgccaaccattgcacagtctctagacgaaccaaatagcgtattcgacccattgtggagcgatttttcaaactttcttgggccattgtcaatggcagatgagaatgatgattacttggcaaatttggaggaaagtatttccgaaaagagccttcagaatgttgtctgggaatag

SEQ ID NO: 7 - protein, 233 aa, Saccharomyces cerevisiae
MYPNLRELNFGRDVLDHSIQSDNETSNLKVNAAHWNLKIKDGRIFFEGPSSSRYIPSNSYSGAKLLETSPSVSHFDELHLRVFQWYFEKMNLSLPLLDETLFFSSLNNSIEHNVQADFAPKCLINCLMAIWLLYGDKKHDKFRLLAIEQVNESMVTGGATLGIIQSFILLSIIEMINGDESSSSDFIARAVAACYHLGLHVTSTDLVRMGKLDYREAKLRDNVFWCCFFFFVF

SEQ ID NO: 8 - DNA, 699 bp, Saccharomyces cerevisiae
aaaaacaaaaaaaaaaaaacaacaccaaaaaacattgtctctaagttttgcctcacggtagtccagttttcccattctgactaaatcagtacttgtcacatgaaggccaagatgataacaggctgcgaccgctcttgcaataaaatccgaagaactagactcatctccatttatcatttctatgatagaaagaagaataaaagattgaataataccaagagtcgctcctccagtaaccatactttcattgacttgctctatagctaataaacggaacttgtcatgtttcttgtccccgtacaaaagccagatggccattaagcaatttatcaaacatttaggtgcgaagtcagcttgaacgttatgttcgatcgaattatttagagagctgaaaaaaagcgtctcgtcgagaagaggtaaggataaattcatcttttcaaaataccattggaaaactctgaggtggagctcatcaaaatgtgacacggagggagaagtttctaacaattttgcgccagaataactatttgaagggatgtatcttgaactcgaaggcccttcaaaaaatattctcccatcctttatcttcaagttccaatgtgcagcgttgactttcaaattagacgtttcattatcagactggatcgagtgatcaagcacatctctcccaaaatttaactctctcaagtttggatacat

SEQ ID NO: 9 - protein, 152 aa, Saccharomyces cerevisiae
MGNGDAEFRKLVKRTVDPARVMIAGINKPSDYEKKFLASHGIRTASPDQVKSGNEEIEKWIKEEGITHLAIHWDLDSLDPKYFRSILFAKPDADEKFFEGVGRGELKLLDVVNLMNRASQHATVVGVGIAEHIPWDSINLKEALAKLPLLSE

SEQ ID NO: 10 - DNA, 459 bp, Saccharomyces cerevisiae
atgggaaatggggacgctgaatttaggaagttggtcaaaagaactgtcgaccctgctagggttatgattgccggtatcaacaaacccagtgattatgaaaagaaattcttagccagtcatggaatcagaaccgcttcccccgatcaagtgaagtcaggtaatgaggaaatagaaaaatggataaaggaagaaggcattacgcacttggctattcactgggatttagattcactggatccgaaatatttccgttctattctttttgccaaaccggatgcagacgagaagtttttcgagggagtaggaagaggtgaactcaaattactagacgttgttaacctcatgaacagagcttcccagcatgctactgtcgttggcgtgggaattgcagagcatattccttgggactcgattaatttgaaggaggccttggcaaagttgcctttgctatcagaatag

SEQ ID NO: 11 - protein, 137 aa, Saccharomyces cerevisiae
MTTYDIDVNKGMNKSLADLVAPWRPKPLKSYCISNTNLIDVVSGATLPGAYIFIENGMISKVEFGSEKPVTVDEGTFEIIDGAGKYVTPGLIDSHVHVASVAGEADLSKLMLIPKSVTLLRIRYTLEAALARGFTTV

SEQ ID NO: 12 - DNA, 412 bp, Saccharomyces cerevisiae
atgacaacttatgacattgatgttaacaaaggaatgaataagtcactggctgacttggtagcaccatggcgcccaaagccacttaaaagttactgcataagtaacaccaacctgatagacgtggtaagtggtgccactctcccaggagcctatattttcatagaaaacggtatgatttctaaggtggaatttggctctgaaaagccagtgaccgttgatgaaggcacttttgaaattattgacggtgccggtaaatacgtcactccaggtttgattgacagtcatgtccacgtcgcgtcagttgcaggagaagcagatttgagcaagttaatgttgataccaaagtcagtcacattgctcagaataagatacactttagaagctgctttggcaagaggtttcacaacggtga

SEQ ID NO: 13 - protein, 134 aa, Saccharomyces cerevisiae
MSASSIVRVVFPQWQGGNNSAYRLGGELLSWLAPKSNSKVIEVDVPATSEKVKLENGIVGREVLIAQAEQVANELEKCTPDKVVVFGGDCLVDLAPFNYLSEKYKEKLGILWIDAHPDVMTKEEYENAHAHVLG

SEQ ID NO: 14 - DNA, 404 bp, Saccharomyces cerevisiae
atgagtgcctccagtatcgtgcgtgtagttttccctcaatggcaaggtggcaataattcagcttaccgtctaggtggtgagcttttatcttggcttgctcctaaatctaattcgaaagttatcgaagtggatgttccagccacttctgaaaaggtgaaactggaaaacggtattgtcggtagagaggttttgattgctcaggctgaacaagtggccaatgaactggaaaaatgcaccccagataaagtggttgtttttgggggagattgtttggttgatcttgctcctttcaactatttaagcgaaaagtacaaggaaaagcttggtattttatggattgacgctcatccggatgttatgacgaaggaagagtatgagaatgcgcatgcccacgttttaggact

SEQ ID NO: 15 - protein, 104 aa, Saccharomyces cerevisiae
MTLAKQACDCCRVRRVKCDGEKPCNRCLQHDLKCTYLQPLRKRGPKNIRSRSLKKIAETQTFSENNNCMTALEISIGIIISYMLFCSVVVTNFRDLFGCYYPCL

SEQ ID NO: 16 - DNA, 315 bp, Saccharomyces cerevisiae
ttataaacaggggtagtagcatccgaaaagatctctgaaattagttactacaacagaacagaataacatgtaagatataatgattcctatagatatttctaaagctgtcatacagttgttgttctcactgaacgtttgcgtttcggcaattttctttaaacttctcgatctaatatttttgggccctctttttctcaaaggttgtaaataagtacatttcaaatcatgctgcagacaacgattacatggcttttcgccgtcacactttactcgacgaacacgacaacaatcgcatgcctgttttgccaaagtcat

SEQ ID NO: 17 - protein, 170 aa, Saccharomyces cerevisiae
MSSFKILKIFYFEKKLDLTNIAEGIELEDFESFTEFIKIENLDQIKKNIYYNNSGYNYTLEFLVYKKNKLFLHSRGLPILYRKPLNMNVEKLHSALYGLITQSSFGPDLIYAYKNDSYIIPQEKILVFSVNIDLEMENENLKIIKIHNENEVYKKILTYNFMEEHKNIYL

SEQ ID NO: 18 - DNA, 510 bp, Saccharomyces cerevisiae
taaatagatattcttatgttcttccataaaattatatgtgagtatttttttatatacctcattttcgttgtgtatttttattatttttagattttcattttccatttcaagatcaatgtttacactgaatactagtatcttttcttgcggtataatgtagctatcatttttatatgcatatatcagatcaggaccaaatgatgattgtgttattaatccatataatgccgaatgcagtttttctacattcatattcaaaggcttcctatataaaattggtaaccctcttgaatggaggaaaagtttatttttcttatagactaaaaactctaaggtgtaattatatccggaattattataataaatattctttttaatttgatccaagttttcaattttaataaattctgtgaaagattcaaaatcttctagctctattccttcagctatatttgttaaatctaattttttttcaaagtaaaaaattttcagtattttgaaggaagacat

SEQ ID NO: 19 - DNA, 4961 bp, Saccharomyces cerevisiae
tgggataggatagtagcaactcttggaggagagcattgtcagttgtccagtctctgaagttaagtagtaagtttgcggagtcaaagggggatggcttttgccatttgtgagagttgtgcggcagcatcttattcaaatagagctgtattctgaagacctcttgtagaacatcatccatactaaaaagtaaatcgtcctgtcccattacgagctgtattagtgctgtgaccctctgtatatttacgttgccatgaagaaggtaatgggcgatattttgatacaattcctgagttgcatgttggattgagtttacgaagggtcgccagacggccagaaacctccaggcggagttaacaactagtaatacggcatccatgtttgcatcagcgccgagcctataccagtcactgagtagacgttttcttgctctttttatgtcctgacttcttttgacgagggggcattctctagagacacaggcagttgcttccagcaactgccgtacggccgttctcatgctgtcgaggattttttttgggacgatattgtcattatagggcagtgtgtgacttatgaattgttgtagaaggacgtctgtgatgttggagatatgtattttgttaactcttcttgagacgatttggccctggatagcgaagcgtgcggttacaaataggtcgtcttgttcaagaaggtaggcgaggacattatctatcagtacaaacatcttagtagtgtctgaggagagggttgattgtttatgtatttttgcgaaatatatatatatatattctacacagatatatacatatttgtttttcgggctcattctttcttctttgccagaggctcaccgctcaagaggtccgctaattctggagcgattgttattgttttttcttttcttcttctattcgaaacccagtttttgatttgaatgcgagataaactggtattcttcattagattctctaggcccttggtatctagatatgggttctcgatgttctttgcaaaccaactttctagtattcggacattttcttttgtaaaccggtgtcctctgtaaggtttagtacttttgtttatcatatcttgagttaccacattaaataccaacccatccgccgatttatttttctgtgtaagttgataattacttctatcgttttctatgctgcgcatttctttgagtaatacagtaatggtagtagtgagttgagatgttgtttgcaacaacttcttctcctcatcactaatcttacggtttttgttggccctagataagaatcctaatatatcccttaattcaacttcttcttctgttgttacactctctggtaacttaggtaaattacagcaaatagaaaagagctttttatttatgtctagtatgctggatttaaactcatctgtgatttgtggatttaaaaggtctttaatgggtattttattcattttttcttgcttatcttcctttttttcttgcccacttctaagctgatttcaatctctcctttatatatatttttaagttccaacattttatgtttcaaaacattaatgatgtctgggttttgtttgggatgcaatttattgcttcccaatgtagaaaagtacatcatatgaaacaacttaaactcttaactacttcttttaaccttcactttttatgaaatgtatcaaccatatataataacttaatagacgacattcacaatatgtttacttcgaagcctgctttcaaaattaagaacaaagcatccaaatcatacagaaacacagcggtttcaaaaaagctgaaagaaaaacgtctagctgagcatgtgaggccaagctgcttcaatattattcgaccactcaagaaagatatccagattcctgttccttcctctcgatttttaaataaaatccaaattcacaggatagcgtctggaagtcaaaatactcagtttcgacagttcaataagacatctataaaatcttcaaagaaatatttaaactcatttatggcttttagagcatattactcacagtttggctccggtgtaaaacaaaatgtcttgtcttctctgctcgctgaagaatggcacgcggacaaaatgcagcacggaatatgggactacttcgcgcaacagtataattttataaaccctggttttggttttgtagagtggttgacgaataattatgctgaagtacgtggtgacggatattgggaagatgtgtttgtacatttggccttatagagtgtggtcgtggcggaggttgtttatctttcgagtactgaatgttgtcagtatagctatcctatttgaaactccccatcgtcttgctcttgttcccaatgtttgtttatacactcatatggctatacccttatctacttgcctcttttgtttatgtctatgtatttgtataaaatatgatattactcagactcaagcaaacaatcaatgctcacacgcggccagggggagcctcgacactagtaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgtttttttttgttttgttctcactgtatttacatttctatttagtatttagttattcatataatcttaacttctcgaggagctccgctcgtccaacgccggcggacctcggaggttgtttatctttcgagtactgaatgttgtcagtatagctatcctatttgaaactccccatcgtcttgctcttgttcccaatgtttgtttatacactcatatggctatacccttatctacttgcctcttttgtttatgtctatgtatttgtataaaatatgatattactcagactcaagcaaacaatcaattcttagcatcattctttgttcttatcttaaccataaacgatcttgatgtgacttttgtaatttgaacgaattggctatacgggacggatgacaaatgcaccattactctaggttgttgttggatcttaacaaaccgtaaaggtaaactgcccatgcggttcacatgacttttgactttcctttgtttgctagttaccttcggcttcacaatttgtttttccacttttctaacaggtttatcacctttcaaacttatctttatcttattcgccttcttgggtgcctccacagtagaggttacttcctttttaatatgtacttttaggatactttcacgctttataaca

SEQ ID NO: 20 - DNA, 4856 bp, Saccharomyces cerevisiae
tgggataggatagtagcaactcttggaggagagcattgtcagttgtccagtctctgaagttaagtagtaagtttgcggagtcaaagggggatggcttttgccatttgtgagagttgtgcggcagcatcttattcaaatagagctgtattctgaagacctcttgtagaacatcatccatactaaaaagtaaatcgtcctgtcccattacgagctgtattagtgctgtgaccctctgtatatttacgttgccatgaagaaggtaatgggcgatattttgatacaattcctgagttgcatgttggattgagtttacgaagggtcgccagacggccagaaacctccaggcggagttaacaactagtaatacggcatccatgtttgcatcagcgccgagcctataccagtcactgagtagacgttttcttgctctttttatgtcctgacttcttttgacgagggggcattctctagagacacaggcagttgcttccagcaactgccgtacggccgttctcatgctgtcgaggattttttttgggacgatattgtcattatagggcagtgtgtgacttatgaattgttgtagaaggacgtctgtgatgttggagatatgtattttgttaactcttcttgagacgatttggccctggatagcgaagcgtgcggttacaaataggtcgtcttgttcaagaaggtaggcgaggacattatctatcagtacaaacatcttagtagtgtctgaggagagggttgattgtttatgtatttttgcgaaatatatatatatatattctacacagatatatacatatttgtttttcgggctcattctttcttctttgccagaggctcaccgctcaagaggtccgctaattctggagcgattgttattgttttttcttttcttcttctattcgaaacccagtttttgatttgaatgcgagataaactggtattcttcattagattctctaggcccttggtatctagatatgggttctcgatgttctttgcaaaccaactttctagtattcggacattttcttttgtaaaccggtgtcctctgtaaggtttagtacttttgtttatcatatcttgagttaccacattaaataccaacccatccgccgatttatttttctgtgtaagttgataattacttctatcgttttctatgctgcgcatttctttgagtaatacagtaatggtagtagtgagttgagatgttgtttgcaacaacttcttctcctcatcactaatcttacggtttttgttggccctagataagaatcctaatatatcccttaattcaacttcttcttctgttgttacactctctggtaacttaggtaaattacagcaaatagaaaagagctttttattcttgatttttgttctttcggggaaactgtataaaacttccaaaaaggaaaagtaaaacaatacatctccttatatcaaagaaaatcaagaaggacaacatggatgatatttgtagtatggcggaaaacataaacagaactctgtttaacattctaggtactgagattgatgaaatcaatctcaatactaataatctttataatgtatgttttcatttcaaggatagcctttgaatcaatttactaacaatacttcagtttataatggaaagtaatttgactaaagtagagcaacatacattacacaaaaatatttctaacaataggttagaaatataccaccacattaaaaaagagaagagcccaaagggaaaatcatcaatatcaccccaagcacgggcatttttagaacaggtttttagaagaaagcaaagccttaattccaaggaaaaagaagaagttgcaaagaaatgtggcattactccacttcaagtaagagtttgggtatgtaatatgagaatcaaacttaaatatatcctatactaacaatttgtagttcataaataaacgtatgagatctaaataaattcgttttcaatgattaaaatagcatagtcgggtttttcttttagtttcagctttccgcaacagtataattttataaaccctggttttggttttgtagagtggttgacgaataattatgctgaagtacgtggtgacggatattgggaagatgtgtttgtacatttggccttatagagtgtggtcgtggcggaggttgtttatctttcgagtactgaatgttgtcagtatagctatcctatttgaaactccccatcgtcttgctcttgttcccaatgtttgtttatacactcatatggctatacccttatctacttgcctcttttgtttatgtctatgtatttgtataaaatatgatattactcagactcaagcaaacaatcaatgctcacacgcggccagggggagcctcgacactagtaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgtttttttttgttttgttctcactgtatttacatttctatttagtatttagttattcatataatcttaacttctcgaggagctccgctcgtccaacgccggcggacctcggaggttgtttatctttcgagtactgaatgttgtcagtatagctatcctatttgaaactccccatcgtcttgctcttgttcccaatgtttgtttatacactcatatggctatacccttatctacttgcctcttttgtttatgtctatgtatttgtataaaatatgatattactcagactcaagcaaacaatcaattcttagcatcattctttgttcttatcttaaccataaacgatcttgatgtgacttttgtaatttgaacgaattggctatacgggacggatgacaaatgcaccattactctaggttgttgttggatcttaacaaaccgtaaaggtaaactgcccatgcggttcacatgacttttgactttcctttgtttgctagttaccttcggcttcacaatttgtttttccacttttctaacaggtttatcacctttcaaacttatctttatcttattcgccttcttgggtgcctccacagtagaggttacttcctttttaatatgtacttttaggatactttcacgctttataaca

(19) United States
(12) Patent Application Publication
(10) Pub. No.: US 2010/0311065 A1
(43) Pub. Date: Dec. 9, 2010

(54) GENETICALLY MODIFIED MICROBES PRODUCING ISOPRENOIDS

(76) Inventors: Jeffrey A. Ubersax, Emeryville, CA (US); Darren M. Platt, Emeryville, CA (US)

Correspondence Address: JONES DAY, 222 EAST 41ST ST, NEW YORK, NY, 10017, US

(21) Appl. No.: 12/791,596

(22) Filed: Jun. 1, 2010

Related U.S. Application Data
(60) Provisional application No. 61/183,029, filed on Jun. 1, 2009.

(51) Int. Cl. C12Q 1/68; C12N 1/19; C12P 5/02; G01N 33/53

(52) U.S. Cl. 435/6; 435/254.2; 435/254.21; 435/167; 435/7.1

(57) ABSTRACT

Provided herein are methods of generating genetically modified yeast cells, e.g., genetically modified diploid and haploid yeast cells, that comprise novel polypeptides, and genetically modified yeast cells that persistently produce isoprenoid compounds in industrial fermentation processes, produced thereby.

This application claims benefit under 35 U.S.C. §119(e) of U.S. Provisional Application No. 61/183,029, filed Jun. 1, 2009, which is hereby incorporated by reference in its entirety.

### 1. FIELD OF THE INVENTION

The compositions and methods provided herein generally relate to the industrial use of microorganisms. In particular, provided herein are genetically modified microorganisms that are significantly more persistent at producing desired products in industrial scale fermentations. More particularly, provided herein are genetically modified yeast cells that persistently produce isoprenoids in industrial scale fermentations, and methods for making and using such genetically modified yeast cells.

### 2. BACKGROUND

Advances in recombinant DNA technology allow for the production of industrially useful substances using genetically modified microorganisms. Among such useful substances are the isoprenoids. Isoprenoids constitute a diverse group of natural compounds that are derived from a single biosynthetic precursor, the five-carbon molecule isopentenyl pyrophosphate (“IPP”). Isoprenoids find commercial application as pharmaceuticals, nutriceuticals, fragrances, flavoring compounds, agricultural pest control agents, and biofuels. Given the low yields achieved by extracting isoprenoids from existing natural sources, genetically modified microorganisms present a promising vehicle for their fermentative production. Genetically modified yeasts in particular have proven useful for fermentative production of commercially useful isoprenoids. Generally, yeasts can grow rapidly and can be cultivated at higher density as compared with bacteria, and do not require an aseptic environment in the industrial setting. Furthermore, yeast cells can be easily separated from culture medium as compared with bacterial cells, greatly simplifying the process for product extraction and purification. Because of these characteristics, yeasts (in particular, genetically modified yeasts harboring recombinant DNA sequences) have been employed as hosts for the production of useful products. However, there exists a continuing need for yeasts that are suitable for industrial applications in general, and for the industrial production of isoprenoids in particular.

### 3. SUMMARY OF THE INVENTION

Provided herein are compositions comprising a genetically modified microbial cell (e.g., a genetically modified Saccharomyces cerevisiae cell) that produces one or more isoprenoid compounds in an industrial fermentation process at greater yield and/or with increased persistence compared to a parent microbial cell that is not genetically modified according to the methods disclosed herein. The genetically modified microbial cell provided herein finds use in industrial applications, e.g., industrial fermentation applications, and can provide the advantage of producing increased levels of commercially useful isoprenoid compounds in industrial fermentation processes.

In one aspect, the present invention provides a genetically modified microbial cell comprising one or more heterologous nucleotide sequences encoding one or more enzymes of the mevalonate-dependent (“MEV”) pathway, and one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17. In some embodiments, the one or more nucleotide sequences are at least 85% identical to a nucleotide sequence selected from the group consisting of SEQ ID NOs: 2, 4, 6, 8, 10, 12, 14, 16, and 18.

In some embodiments, the one or more heterologous nucleotide sequences encode an enzyme that can convert HMG-CoA into mevalonate, e.g., a HMG-CoA reductase. In some embodiments, the one or more heterologous nucleotide sequences encode an enzyme that can convert mevalonate into mevalonate 5-phosphate, e.g., a mevalonate kinase. In some embodiments, the genetically modified microbial cell comprises one or more heterologous nucleotide sequences encoding an enzyme that can convert HMG-CoA into mevalonate and an enzyme that can convert mevalonate into mevalonate 5-phosphate.

In some embodiments, the genetically modified microbial cell further comprises a heterologous nucleotide sequence encoding an enzyme that can convert IPP generated via the MEV pathway into its isomer, dimethylallyl pyrophosphate (“DMAPP”), e.g., an IPP isomerase. In some embodiments, the genetically modified microbial cell further comprises a heterologous nucleotide sequence encoding an enzyme that can condense IPP and/or DMAPP molecules to form polyprenyl compounds containing more than five carbons. In some embodiments, the genetically modified microbial cell further comprises a heterologous nucleotide sequence encoding an enzyme that can modify IPP or a polyprenyl to form and an isoprenoid compound.

In some embodiments, the genetically modified microbial cell further comprises one or more heterologous nucleotide sequences encoding one or more proteins that increase flocculation. In some embodiments, the genetically modified microbial cell of the invention comprises one or more heterologous nucleotide sequences encoding one or more flocculation proteins selected from the group consisting of Flo1p, Flo5p, Flo8p, Flo9p, Flo10p, and Flo11p.

In some embodiments, the genetically modified microbial cell is a haploid microbial cell. In other embodiments, the genetically modified microbial cell is a diploid microbial cell. In some embodiments, the genetically modified diploid microbial cell is heterozygous. In other embodiments, the genetically modified diploid microbial cell is homozygous other than for its mating type allele.

In some embodiments, the genetically modified microbial cell of the invention is sporulation impaired and/or endogenous mating impaired, and thus poses reduced risk of: (1) dissemination in nature; and (2) exchange of genetic material between the genetically modified microbial cell and a wild-type microbe that is not compromised in its ability to disseminate in nature.

In some embodiments, the genetically modified microbial cell is a haploid yeast cell in which one or more of the following pheromone response genes: STE5, STE4, STE18, STE12, STE7, and STE11, and/or one or more of the following sporulation genes: IME1, IME2, NDT80, SPO11, SPO20; AMA1, HOP2, and SPO21, are functionally disrupted. In some embodiments, the genetically modified microbial cell is a haploid yeast cell in which the IME1 gene and the STE5 gene are functionally disrupted. In some embodiments, the genetically modified microbial cell is a haploid yeast cell in which the IME1 gene and the STE5 gene are functionally disrupted and that comprises one or more heterologous nucleotide sequences encoding an enzyme that can convert HMG-CoA into mevalonate and an enzyme that can convert mevalonate into mevalonate 5-phosphate.

In some embodiments, the genetically modified haploid yeast cell comprises one or more recombinant plasmids encoding the one or more pheromone response genes that are functionally disrupted in said haploid yeast cell. In some embodiments, the genetically modified yeast cell is a heterothallic (ho) haploid cell. In some embodiments, the genetically modified haploid cell comprises a recombinant plasmid encoding a homothallism (HO) protein.

In some embodiments, the genetically modified microbial cell is a diploid yeast cell in which both copies of one or more of the following pheromone response genes: STE5, STE4, STE18, STE12, STE7, and STE11, and/or both copies of one or more of the following sporulation genes: IME1, IME2, NDT80, SPO11, SPO20, AMA1, HOP2, and SPO21, are functionally disrupted. In some embodiments, the genetically modified microbial cell is a diploid yeast cell in which both copies of the IME1 gene and both copies of the STE5 gene are functionally disrupted, and that comprises one or more heterologous nucleotide sequences encoding an enzyme that can convert HMG-CoA into mevalonate and an enzyme that can convert mevalonate into mevalonate 5-phosphate.

In some embodiments, the genetically modified yeast cell useful for the practice of the methods provided herein is a Saccharomyces cerevisiae cell. In particular embodiments, the Saccharomyces cerevisiae cell is a PE-2 cell.

In another aspect, provided herein is a method for generating a genetically modified yeast cell of the invention. In some embodiments, the method comprises: (a) obtaining a first genetically modified haploid yeast cell, wherein the first genetically modified haploid yeast cell is sporulation and endogenous mating impaired, and comprises one or more heterologous nucleotide sequences encoding one or more enzymes of the MEV pathway, and one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17; (b) obtaining a second genetically modified haploid yeast cell, wherein the second genetically modified haploid yeast cell is sporulation and endogenous mating impaired, is of the opposite mating type as the first genetically modified haploid yeast cell, and comprises one or more heterologous nucleotide sequences encoding said one or more enzymes of the MEV pathway and said one or more nucleotide sequences encoding said one or more polypeptides having an amino acid sequence that is at least 80% identical to an amino acid sequence selected from the group consisting of SEQ ID NOs: 1, 3, 5, 7, 9, 11, 13, 15, and 17; (c) transforming each of the first and the second genetically modified haploid yeast cells with one or more plasmids encoding a protein capable of complementing the endogenous mating impairment of said first and second genetically modified haploid yeast cells; (d) mating the first genetically modified haploid yeast cell with the second genetically modified haploid yeast cell, thereby forming a genetically modified diploid yeast cell; and (e) eliminating the one or more plasmids from the genetically modified diploid yeast cell, wherein the resulting genetically modified diploid yeast cell is sporulation and endogenous mating impaired and comprises two copies of said one or more heterologous nucleotide sequences encoding said one or more enzymes of the MEV pathway and two copies of said one or more nucleotide sequences encoding said one or more polypeptides having an amino acid sequence that is at least 80% identical to an amino acid sequence selected from the group consisting of SEQ ID NOs: 1, 3, 5, 7, 9, 11, 13, 15, and 17.

In some embodiments, the first genetically modified haploid yeast cell and the second genetically modified haploid yeast cell are endogenous mating impaired due to a functional disruption of one or more pheromone response genes. In some embodiments, step (c) of the method of the invention comprises transforming each of the first and the second genetically modified haploid yeast cells with one or more plasmids encoding a functional copy of the one or more pheromone response genes that are functionally disrupted in said first and second genetically modified haploid yeast cells. In certain embodiments, the first and second genetically modified haploid yeast cells are haploid yeast cells that are endogenous mating impaired due to a functional disruption of the STE5 gene.

In some embodiments, the first genetically modified haploid yeast cell and the second genetically modified haploid yeast cell are sporulation impaired due to a functional disruption of one or more sporulation genes. In some embodiments, the first and second genetically modified haploid yeast cells are haploid yeast cells that are sporulation impaired due to a functional disruption of the IME1 gene. In particular embodiments, the first and second genetically modified haploid yeast cells are haploid yeast cells that are endogenous mating impaired due to a functional disruption of the STE5 gene, and are sporulation impaired due to a functional disruption of the IME1 gene.

In some embodiments, the second genetically modified haploid yeast cell is obtained by inducing a mating type switch in a population of the first genetically modified haploid yeast cell. In some embodiments, the first genetically modified haploid yeast cell is a heterothallic (ho) haploid Saccharomyces cerevisiae cell, and said population of heterothallic (ho) haploid Saccharomyces cerevisiae cell is induced to switch mating type by transforming said heterothallic (ho) haploid Saccharomyces cerevisiae cell with a plasmid encoding a homothallism (HO) protein, wherein expression of the HO protein induces a mating type switch in the haploid Saccharomyces cerevisiae cell to yield the second genetically modified haploid Saccharomyces cerevisiae cell.

In other embodiments, the second genetically modified haploid yeast cell is obtained by changing the mating type locus in the first genetically modified haploid yeast cell using recombinant DNA technology. In some embodiments, the first genetically modified haploid yeast cell is transformed with an integration construct that comprises as an integrating sequence a nucleotide sequence that encodes a mating type other than the mating type of the first genetically modified haploid yeast cell, flanked by homologous sequences that are homologous to nucleotide sequences that flank the mating type locus in the first genetically modified haploid yeast cell. In some embodiments, the integration construct is used to switch the mating type of the first genetically modified haploid yeast cell from a to alpha using an integration construct encoding encoding the alpha mating type (MAT alpha). In some embodiments, the integration construct comprises SEQ ID NO: 19. In other embodiments, the integration construct is used to switch the mating type of the first genetically modified haploid yeast cell from alpha to a using an integration construct encoding encoding the a mating type (MAT A). In some embodiments, the integration construct comprises SEQ ID NO: 20.

In another aspect, provided herein is a method for generating a genetically modified heterothallic (ho) diploid yeast cell that lacks sporulation and endogenous mating capability, the method comprising: (a) obtaining a first genetically modified heterothallic haploid yeast cell, wherein the first genetically modified heterothallic haploid yeast cell is sporulation and endogenous mating impaired and comprises one or more heterologous nucleotide sequences encoding one or more enzymes of the MEV pathway and one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17; (b) transforming the first genetically modified heterothallic haploid yeast cell with a plasmid encoding a homothallism (HO) protein to yield a first genetically modified haploid yeast cell, wherein expression of the HO protein induces a mating-type switch in the first genetically modified haploid yeast cell, whereby a second genetically modified haploid yeast cell is obtained, wherein the second genetically modified haploid yeast cell is sporulation and endogenous mating impaired, is of the opposite mating type as the first genetically modified haploid yeast cell, and comprises one or more heterologous nucleotide sequences encoding said one or more enzymes of the MEV pathway and said one or more nucleotide sequences encoding said one or more polypeptides having an amino acid sequence that is at least 80% identical to an amino acid sequence selected from the group consisting of SEQ ID NOs: 1, 3, 5, 7, 9, 11, 13, 15, and 17; (c) transforming each of the first and the second genetically modified haploid yeast cells with a plasmid encoding the one or more pheromone response proteins that are functionally disrupted in said first and second haploid yeast cell; (d) mating the first genetically modified haploid yeast cell with the second genetically modified haploid yeast cell, thereby forming a genetically modified diploid yeast cell that is homozygous other than for its mating type allele; and (e) eliminating any plasmids from the genetically modified diploid yeast cell to yield a genetically modified heterothallic diploid yeast cell, wherein the resulting genetically modified heterothallic diploid yeast cell is sporulation and endogenous mating impaired and comprises two copies of said one or more heterologous nucleotide sequences encoding said one or more enzymes of the MEV pathway and two copies of said one or more nucleotide sequences encoding said one or more polypeptides having an amino acid sequence that is at least 80% identical to an amino acid sequence selected from the group consisting of SEQ ID NOs: 1, 3, 5, 7, 9, 11, 13, 15, and 17.

In another aspect, provided herein is a method for producing an isoprenoid compound comprising: (a) obtaining a plurality of genetically modified yeast cells comprising one or more heterologous nucleotide sequences encoding one or more enzymes of the MEV pathway, and one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17; (b) culturing said genetically modified yeast cells in a medium comprising a carbon source under conditions suitable for making the isoprenoid compound; and (c) recovering the isoprenoid compound from the medium.

In some embodiments, the isoprenoid compound is a C₅ isoprenoid. In other embodiments, the isoprenoid compound is a C₁₀ isoprenoid. In other embodiments, the isoprenoid compound is a C₁₅ isoprenoid. In other embodiments, the isoprenoid compound is a C₂₀ isoprenoid. In yet other examples, the isoprenoid compound is a C₂₀₊ isoprenoid. In some embodiments, the isoprenoid compound is selected from the group consisting of abietadiene, amorphadiene, carene, α-farnesene, β-farnesene, farnesol, geraniol, geranylgeraniol, isoprene, linalool, limonene, myrcene, nerolidol, ocimene, patchoulol, β-pinene, sabinene, γ-terpinene, terpinolene and valencene.

In another aspect, provided herein is a method for detecting in a biological sample the presence or absence of a genetically modified microbial cell comprising one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17. In some embodiments, the method comprises: (a) obtaining a biological sample (e.g., a yeast cell and a population of yeast cells); (b) contacting the biological sample with a first compound or agent capable of interacting with a target molecule, wherein the target molecule is either a nucleic acid encoding a polypeptide having an amino acid sequence that is at least 80% identical to an amino acid sequence selected from the group consisting of SEQ ID NOs: 1, 3, 5, 7, 9, 11, 13, 15, and 17, or a polypeptide having an amino acid sequence that is at least 80% identical to an amino acid sequence selected from the group consisting of SEQ ID NOs: 1, 3, 5, 7, 9, 11, 13, 15, and 17; and (c) detecting said interaction of the first compound or agent with said target molecule, wherein detection of said interaction of the first compound or agent with the target molecule indicates the presence in the biological sample of a genetically modified microbial cell comprising one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17.

### 4. BRIEF DESCRIPTION OF THE FIGURES

FIG. 1 provides a schematic representation of the mevalonate (“MEV”) pathway for the production of isopentenyl diphosphate (“IPP”).

FIG. 2 provides a schematic representation of the conversion of IPP and dimethylallyl pyrophosphate (“DMAPP”) to geranyl pyrophosphate (“GPP”), farnesyl pyrophosphate (“FPP”), and geranylgeranyl pyrophosphate (“GGPP”).

FIG. 3 provides a structure of the Phase I disruption construct and of the target locus after integration of the disrupting sequence by homologous recombination.

FIG. 4 provides a structure of the Phase II disruption construct and of the target locus after integration of the disrupting sequence by homologous recombination.

FIG. 5 provides a structure of the Phase III disruption construct and of the target locus after integration of the disrupting sequence by homologous recombination.

FIG. 6 provides a structure of the Phase I marker recycling construct and of the target locus after integration of the construct by homologous recombination.

FIG. 7 provides a structure of the Phase II marker recycling construct and of the target locus after integration of the construct by homologous recombination.

FIG. 8 provides a structure of the Phase III marker recycling construct and of the target locus after integration of the construct by homologous recombination.

FIG. 9 provides a structure of the STE5 disruption construct and of the target locus after integration of the disrupting sequence by homologous recombination.

FIG. 10 provides a structure of the IME1 disruption construct and of the target locus after integration of the disrupting sequence by homologous recombination.

FIG. 11 provides a comparison of mating capability of genetically modified endogenous mating impaired haploid Y1915 cells and genetically modified endogenous mating competent Y1912 cells.

FIG. 12 provides a comparison of sporulation capability of genetically modified sporulation and endogenous mating impaired diploid Y1979 cells and genetically unmodified sporulation and endogenous mating competent Y1198 cells.

FIG. 13 provides a comparison of survival in soil of genetically modified sporulation and endogenous mating impaired diploid Y1979 cells and genetically unmodified sporulation and endogenous mating competent Y1198 cells.

### 5. DETAILED DESCRIPTION OF THE EMBODIMENTS

### 5.1 Definitions

As used herein, the term “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); or (c) be naturally found in the host cell but positioned outside of its natural locus.

As used herein, to “functionally disrupt” or a “functional disruption” of a target gene, e.g., a pheromone response gene or a sporulation gene, means that the target gene is altered in such a way as to decrease in the host cell the activity of the protein encoded by the target gene. In some embodiments, the activity of the protein encoded by the target gene is eliminated in the host cell. In other embodiments, the activity of the protein encoded by the target gene is decreased in the host cell. Functional disruption of the target gene may be achieved by deleting all or a part of the gene so that gene expression is eliminated or reduced or so that the activity of the gene product is eliminated or reduced. Functional disruption of the target gene may also be achieved by mutating a regulatory element of the gene, e.g., the promoter of the gene so that expression is eliminated or reduced, or by mutating the coding sequence of the gene so that the activity of the gene product is eliminated or reduced. In some embodiments, functional disruption of the target gene results in the removal of the complete open reading frame of the target gene.

As used herein, “endogenous mating” and “endogenous mating capability” refer to the ability of haploid microbial cells of opposite mating types, i.e. mating types a and alpha, to form a diploid cell in the absence of heterologous gene expression, e.g., expression of a heterologous copy of a pheromone response gene or of any gene capable of inducing mating among such haploids.

As used herein, “endogenous mating impaired” refers to a reduction in the endogenous mating capability of a microbial cell sufficient to inhibit mating within a population of haploids of such a microbial cell, relative to a population of wild-type haploid microbial cells. In some embodiments, inhibition comprises a reduction of at least 10%, 20%, 30%, 40%, 50%, 60%, 65%, 70%, 75% 80%, 85%, 90%, or 95% in the mating rate of a population of haploid microbial cells relative to the mating rate of a population of wild-type haploid microbial cells.

As used herein, “sporulation impaired” refers to a reduction in the sporulation activity of a diploid microbial cell sufficient to inhibit sporulation within a population of diploids of such a microbial cell, relative to a population of wild-type diploid microbial cells. In some embodiments, inhibition comprises a reduction of at least 10%, 20%, 30%, 40%, 50%, 60%, 65%, 70%, 75% 80%, 85%, 90%, or 95% in the sporulation rate of a population of diploid microbial cells relative to the sporulation rate of a population of wild-type diploid microbial cells.

As used herein, the term “complementing” in the context of a gene refers to a gene that has the facility to replace the function of a functionally disrupted gene, e.g., a functionally disrupted sporulation or pheromone response gene. In some embodiments, the mechanism of function between the complementing gene and the disrupted gene need not be identical. In some embodiments, a target gene, e.g., a sporulation gene or a pheromone response gene, that has been functionally disrupted can be complemented by a heterologous gene that either produces a protein homologous to the protein encoded by the disrupted gene or a protein that provides a phenotype that permits, for example, sporulation or mating by an alternative mechanism.

As used herein, the term “persistent” in the context of production of an isoprenoid by a genetically modified microbial cell refers to the ability of the genetically modified microbial cell to produce an isoprenoid compound over longer time spans in an industrial fermentation, compared to a non-genetically modified parent microbial cell.

As used herein, the term “parent” refers to a microbial cell that does not comprise all of the genetic modifications of a genetically modified microbial cell as described herein, but that serves as the starting point for introduction of said genetic modifications, which leads to the generation of such a genetically modified microbial cell.

### 5.2 Genetically Modified Microbial Cells and Methods for Making and Detecting the Same

Provided herein are compositions comprising a genetically modified microbial cell (e.g., a genetically modified Saccharomyces cerevisiae cell) that produces one or more isoprenoid compounds, and methods and materials for generating such compositions. The genetically modified microbial cell of the invention produces the one or more isoprenoid compounds in an industrial fermantion process at greater yield and/or with increased persistence compared to a parent microbial cell that is not genetically modified according to the methods disclosed herein.

Saccharomyces cerevisiae strain PE-2 has been extensively used in the Brazilian fuel ethanol industry. It was originally selected in an ethanol distillery in 1994 based on its marked capacity to compete with wild-type yeast strains, and its ability to survive and dominate during industrial fermentations. Compared to wild-type yeast strains, the PE-2 strain can better tolerate the severe cell recycling procedures and fermentation conditions that are commonly employed in industrial processes, which may include high ethanol concentration, high cell density, high temperature, osmotic stress, low pH, and sulfite and bacterial contamination. See, for example, Basso et al. (2008) FEMS Yeast Research 8(7):1155-1163. PE-2 cells are characterized as comprising nucleotide sequences disclosed herein as SEQ ID NOs: 2, 4, 6, 8, 10, 12, 14, 16, and 18, which do not appear to be present in yeast cells that are less suitable for industrial scale fermentation. Moreover, the ability of PE-2 cells to better persist in an industrial scale fermentation process may be dependent on the function of polypeptides that are encoded by these nucleotide sequences and that have amino acid sequences disclosed herein as SEQ ID NOs: 1, 3, 5, 7, 9, 11, 13, 15, and 17.

In one aspect, the present invention provides a genetically modified microbial cell comprising one or more heterologous nucleotide sequences encoding one or more enzymes of the mevalonate-dependent (“MEV”) pathway, and one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17. In some embodiments, the one or more nucleotide sequences are at least 85% identical to a nucleotide sequence selected from the group consisting of SEQ ID NOs: 2, 4, 6, 8, 10, 12, 14, 16, and 18.

In some embodiments, the one or more heterologous nucleotide sequences encode an enzyme that can condense two molecules of acetyl-coenzyme A to form acetoacetyl-CoA, e.g., an acetyl-CoA thiolase. In some embodiments, the one or more heterologous nucleotide sequences encode an enzyme that can condense acetoacetyl-CoA with another molecule of acetyl-CoA to form 3-hydroxy-3-methylglutaryl-CoA (HMG-CoA), e.g., a HMG-CoA synthase. In some embodiments, the one or more heterologous nucleotide sequences encode an enzyme that can convert HMG-CoA into mevalonate, e.g., a HMG-CoA reductase. In some embodiments, the one or more heterologous nucleotide sequences encode an enzyme that can convert mevalonate into mevalonate 5-phosphate, e.g., a mevalonate kinase. In some embodiments, the one or more heterologous nucleotide sequences encode an enzyme that can convert mevalonate 5-phosphate into mevalonate 5-pyrophosphate, e.g., a phosphomevealonate kinase. In some embodiments, the one or more heterologous nucleotide sequences encode an enzyme that can convert mevalonate 5-pyrophosphate into IPP, e.g., a mevalonate pyrophosphate decarboxylase.

In some embodiments, the heterologous nucleotide sequence encodes an enzyme that can convert HMG-CoA into mevalonate, and the nucleotide sequence encodes a polypeptide that has an amino acid sequence that is at least 80% identical to SEQ ID NO: 1. In some embodiments, the heterologous nucleotide sequence encodes an enzyme that can convert HMG-CoA into mevalonate, and the nucleotide sequence encodes a polypeptide that has an amino acid sequence that is at least 80% identical to SEQ ID NO: 3. In some embodiments, the heterologous nucleotide sequence encodes an enzyme that can convert HMG-CoA into mevalonate, and the nucleotide sequence encodes a polypeptide that has an amino acid sequence that is at least 80% identical to SEQ ID NO: 5. In some embodiments, the heterologous nucleotide sequence encodes an enzyme that can convert HMG-CoA into mevalonate, and the nucleotide sequence encodes a polypeptide that has an amino acid sequence that is at least 80% identical to SEQ ID NO: 7. In some embodiments, the heterologous nucleotide sequence encodes an enzyme that can convert HMG-CoA into mevalonate, and the nucleotide sequence encodes a polypeptide that has an amino acid sequence that is at least 80% identical to SEQ ID NO: 9. In some embodiments, the heterologous nucleotide sequence encodes an enzyme that can convert HMG-CoA into mevalonate, and the nucleotide sequence encodes a polypeptide that has an amino acid sequence that is at least 80% identical to SEQ ID NO: 11. In some embodiments, the heterologous nucleotide sequence encodes an enzyme that can convert HMG-CoA into mevalonate, and the nucleotide sequence encodes a polypeptide that has an amino acid sequence that is at least 80% identical to SEQ ID NO: 13. In some embodiments, the heterologous nucleotide sequence encodes an enzyme that can convert HMG-CoA into mevalonate, and the nucleotide sequence encodes a polypeptide that has an amino acid sequence that is at least 80% identical to SEQ ID NO: 15. In some embodiments, the heterologous nucleotide sequence encodes an enzyme that can convert HMG-CoA into mevalonate, and the nucleotide sequence encodes a polypeptide that has an amino acid sequence that is at least 80% identical to SEQ ID NO: 17. In some embodiments, the one or more heterologous nucleotide sequences encode an enzyme that can convert HMG-CoA into mevalonate, and the one or more nucleotide sequences are at least 85% identical to a nucleotide sequence selected from the group consisting of SEQ ID NOs: 2, 4, 6, 8, 10, 12, 14, 16, and 18.

In some embodiments, the heterologous nucleotide sequence encodes an enzyme that can convert mevalonate into mevalonate 5-phosphate, and the nucleotide sequence encodes a polypeptide that has an amino acid sequence that is at least 80% identical to SEQ ID NO: 1. In some embodiments, the heterologous nucleotide sequence encodes an enzyme that can convert mevalonate into mevalonate 5-phosphate, and the nucleotide sequence encodes a polypeptide that has an amino acid sequence that is at least 80% identical to SEQ ID NO: 3. In some embodiments, the heterologous nucleotide sequence encodes an enzyme that can convert mevalonate into mevalonate 5-phosphate, and the nucleotide sequence encodes a polypeptide that has an amino acid sequence that is at least 80% identical to SEQ ID NO: 5. In some embodiments, the heterologous nucleotide sequence encodes an enzyme that can convert mevalonate into mevalonate 5-phosphate, and the nucleotide sequence encodes a polypeptide that has an amino acid sequence that is at least 80% identical to SEQ ID NO: 7. In some embodiments, the heterologous nucleotide sequence encodes an enzyme that can convert mevalonate into mevalonate 5-phosphate, and the nucleotide sequence encodes a polypeptide that has an amino acid sequence that is at least 80% identical to SEQ ID NO: 9. In some embodiments, the heterologous nucleotide sequence encodes an enzyme that can convert mevalonate into mevalonate 5-phosphate, and the nucleotide sequence encodes a polypeptide that has an amino acid sequence that is at least 80% identical to SEQ ID NO: 11. In some embodiments, the heterologous nucleotide sequence encodes an enzyme that can convert mevalonate into mevalonate 5-phosphate, and the nucleotide sequence encodes a polypeptide that has an amino acid sequence that is at least 80% identical to SEQ ID NO: 13. In some embodiments, the heterologous nucleotide sequence encodes an enzyme that can convert mevalonate into mevalonate 5-phosphate, and the nucleotide sequence encodes a polypeptide that has an amino acid sequence that is at least 80% identical to SEQ ID NO: 15. In some embodiments, the heterologous nucleotide sequence encodes an enzyme that can convert mevalonate into mevalonate 5-phosphate, and the nucleotide sequence encodes a polypeptide that has an amino acid sequence that is at least 80% identical to SEQ ID NO: 17. In some embodiments, the one or more heterologous nucleotide sequences encode an enzyme that can convert mevalonate into mevalonate 5-phosphate, and the one or more nucleotide sequences are at least 85% identical to a nucleotide sequence selected from the group consisting of SEQ ID NOs: 2, 4, 6, 8, 10, 12, 14, 16, and 18.

In some embodiments, the genetically modified microbial cell comprises one or more heterologous nucleotide sequences encoding more than one enzyme of the MEV pathway. In some embodiments, the genetically modified microbial cell comprises one or more heterologous nucleotide sequences encoding two enzymes of the MEV pathway. In some embodiments, the genetically modified microbial cell comprises one or more heterologous nucleotide sequences encoding an enzyme that can convert HMG-CoA into mevalonate and an enzyme that can convert mevalonate into mevalonate 5-phosphate. In some embodiments, the genetically modified microbial cell comprises one or more heterologous nucleotide sequences encoding three enzymes of the MEV pathway. In some embodiments, the genetically modified microbial cell comprises one or more heterologous nucleotide sequences encoding four enzymes of the MEV pathway. In some embodiments, the genetically modified microbial cell comprises one or more heterologous nucleotide sequences encoding five enzymes of the MEV pathway. In some embodiments, the genetically modified microbial cell comprises one or more heterologous nucleotide sequences encoding six enzymes of the MEV pathway.

In some embodiments, the genetically modified microbial cell comprises more than one nucleotide sequence encoding more than one polypeptide having an amino acid sequence that is at least 80% identical to an amino acid sequence selected from the group consisting of SEQ ID NOs: 1, 3, 5, 7, 9, 11, 13, 15, and 17. In some embodiments, the genetically modified microbial cell comprises two nucleotide sequences encoding two or more polypeptides having an amino acid sequence that is at least 80% identical to an amino acid sequence selected from the group consisting of SEQ ID NOs: 1, 3, 5, 7, 9, 11, 13, 15, and 17. In some embodiments, the genetically modified microbial cell comprises three nucleotide sequences encoding three or more polypeptides having an amino acid sequence that is at least 80% identical to an amino acid sequence selected from the group consisting of SEQ ID NOs: 1, 3, 5, 7, 9, 11, 13, 15, and 17. In some embodiments, the genetically modified microbial cell comprises four nucleotide sequences encoding four or more polypeptides having an amino acid sequence that is at least 80% identical to an amino acid sequence selected from the group consisting of SEQ ID NOs: 1, 3, 5, 7, 9, 11, 13, 15, and 17. In some embodiments, the genetically modified microbial cell comprises eight nucleotide sequences encoding eight polypeptides having amino acid sequences that are at least 80% identical to SEQ ID NOs: 1, 3, 5, 7, 9, 11, 13, 15, and 17.

In some embodiments, the genetically modified microbial cell further comprises a heterologous nucleotide sequence encoding an enzyme that can convert IPP generated via the MEV pathway into its isomer, dimethylallyl pyrophosphate (“DMAPP”), e.g., an IPP isomerase. In some embodiments, the genetically modified microbial cell further comprises a heterologous nucleotide sequence encoding an enzyme that can condense IPP and/or DMAPP molecules to form polyprenyl compounds containing more than five carbons, such as, for example, geranyl pyrophosphate (“GPP”), farnesyl pyrophosphate (“FPP”), and geranylgeranyl pyrophosphate (“GGPP”), e.g., a GPP synthase, a FPP synthase, or a GGPP synthase. In some embodiments, the genetically modified microbial cell further comprises a heterologous nucleotide sequence encoding an enzyme that can modify IPP or a polyprenyl to form and isoprenoid compound, such as, for example, a hemiterpene, a monoterpene, a sesquiterpene, a diterpene, a triterpene, a tetraterpene, a polyterpene, a steroid compound, a carotenoid, or a modified isoprenoid compounds.

In some embodiments, the genetically modified microbial cell is a haploid microbial cell. In other embodiments, the genetically modified microbial cell is a diploid microbial cell. In some embodiments, the genetically modified diploid microbial cell is heterozygous. In other embodiments, the genetically modified diploid microbial cell is homozygous other than for its mating type allele (i.e., if the genetically modified diploid microbial cell should sporulate, the resulting four haploid microbial cells would be genetically identical except for their mating type allele, which in two of the haploid cells would be mating type a and in the other two haploid cells would be mating type alpha).

In some embodiments, the genetically modified microbial cell further comprises one or more heterologous nucleotide sequences encoding one or more proteins that increase flocculation. Flocculation is the asexual, reversible, and calcium-dependent aggregation of microbial cells to form flocs containing large numbers of cells that rapidly sediment to the bottom of the liquid growth substrate. Flocculation is of significance in industrial fermentations of yeast, e.g., for the production of bioethanol, wine, beer, and other products, because it greatly simplifies the processes for separating the suspended yeast cells from the fermentation products produced therefrom in the industrial fermentation. The separation may be achieved by centrifugation or filtration, but separation by these methods is time-consuming and expensive. Clarification can be alternatively achieved by natural settling of the microbial cells. Although single microbial cells tend to settle over time, natural settling becomes a viable option in industrial processes only when cells aggregate (i.e., flocculate). Recent studies demonstrate that the flocculation behavior of yeast cells can be tightly controlled and fine-tuned to satisfy specific industrial requirements (see, e.g., Governder et al., Appl Environ Microbiol. 74(19):6041-52 (2008), the contents of which are hereby incorporated by reference in their entirety). Flocculation behavior of yeast cells is dependent on the function of specific flocculation proteins, including, but not limited to, products of the FLO1, FLO5, FLO8, FLO9, FLO10, and FLO11 genes. Thus, in some embodiments, the genetically modified microbial cell of the invention comprises one or more heterologous nucleotide sequences encoding one or more flocculation proteins selected from the group consisting of Flo1p, Flo5p, Flo8p, Flo9p, Flo10p, and Flo11p.

In some embodiments, the genetically modified microbial cell of the invention is sporulation impaired and/or endogenous mating impaired. A sporulation and/or endogenous mating impaired genetically modified microbial cell poses reduced risk of: (1) dissemination in nature; and (2) exchange of genetic material between the genetically modified microbial cell and a wild-type microbe that is not compromised in its ability to disseminate in nature. In yeast, the ability of diploid microbial cells to sporulate, and of haploid microbial cells to mate, is dependent on the function of specific gene products. Among these in yeast are products of sporulation genes, such as of the IME1, IME2, NDT80, SPO11, SPO20, AMA1, HOP2, and SPO21 genes, and products of pheromone response genes, such as of the STE5, STE4, STE18, STE12, STE7 and STE11 genes.

In some embodiments, the genetically modified microbial cell is a haploid yeast cell in which one or more of the following pheromone response genes is functionally disrupted: STE5, STE4, STE18, STE12, STE7, and STE11. In some embodiments, the genetically modified microbial cell is a haploid yeast cell in which one or more of the following sporulation genes is functionally disrupted: IME1, IME2, NDT80, SPO11, SPO20, AMA1, HOP2, and SPO21. In some embodiments, the genetically modified microbial cell is a haploid yeast cell in which one or more of the following pheromone response genes: STE5, STE4, STE18, STE12, STE7, and STE11, and one or more of the following sporulation genes: IME1, IME2, NDT80, SPO11, SPO20, AMA1, HOP2, and SPO21, are functionally disrupted. In some embodiments, the genetically modified microbial cell is a haploid yeast cell in which the IME1 gene and the STE5 gene are functionally disrupted. In some embodiments, the genetically modified microbial cell is a haploid yeast cell in which the IME1 gene and the STE5 gene are functionally disrupted and that comprises a heterologous nucleotide sequence encoding an enzyme that can convert HMG-CoA into mevalonate. In some embodiments, the genetically modified microbial cell is a haploid yeast cell in which the IME1 gene and the STE5 gene are functionally disrupted, and that comprises a heterologous nucleotide sequence encoding an enzyme that can convert mevalonate into mevalonate 5-phosphate.

In some embodiments, the genetically modified microbial cell is a diploid yeast cell in which both copies of one or more of the following pheromone response genes are functionally disrupted: STE5, STE4, STE18, STE12, STE7, and STE11. In some embodiments, the genetically modified microbial cell is a diploid yeast cell in which both copies of one or more of the following sporulation genes are functionally disrupted: IME1, IME2, NDT80, SPO11, SPO20, AMA1, HOP2, and SPO21. In some embodiments, the genetically modified microbial cell is a diploid yeast cell in which both copies of one or more of the following pheromone response genes: STE5, STE4, STE18, STE12, STE7, and STE11, and both copies of one or more of the following sporulation genes: IME1, IME2, NDT80, SPO11, SPO20, AMA1, HOP2, and SPO21, are functionally disrupted. In some embodiments, the genetically modified microbial cell is a diploid yeast cell in which both copies of the IME1 gene and both copies of the STE5 gene are functionally disrupted. In some embodiments, the genetically modified microbial cell is a diploid yeast cell in which both copies of the IME1 gene and both copies of the STE5 gene are functionally disrupted, and that comprises a heterologous nucleotide sequence encoding an enzyme that can convert HMG-CoA into mevalonate. In some embodiments, the genetically modified microbial cell is a diploid yeast cell in which both copies of the IME1 gene and both copies of the STE5 gene are functionally disrupted, and that comprises a heterologous nucleotide sequence encoding an enzyme that can convert mevalonate into mevalonate 5-phosphate.

In some embodiments, the genetically modified microbial cell of the invention comprises a functional disruption in one or more biosynthesis genes, wherein said genetically modified microbial cell is auxotrophic as a result of said disruption. In some embodiments, the genetically modified microbial cell of the invention comprises one or more selectable marker genes. In some embodiments, the genetically modified microbial cell of the invention does not comprise a heterologous nucleotide sequence that confers resistance to an antibiotic compound.

In another aspect, provided herein is a method for generating a genetically modified yeast cell of the invention. In some embodiments, the method comprises: (a) obtaining a first genetically modified haploid yeast cell, wherein the first genetically modified haploid yeast cell is sporulation and endogenous mating impaired, and comprises one or more heterologous nucleotide sequences encoding one or more enzymes of the MEV pathway, and one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17; (b) obtaining a second genetically modified haploid yeast cell, wherein the second genetically modified haploid yeast cell is sporulation and endogenous mating impaired, is of the opposite mating type as the first genetically modified haploid yeast cell, and comprises one or more heterologous nucleotide sequences encoding said one or more enzymes of the MEV pathway and said one or more nucleotide sequences encoding said one or more polypeptides having an amino acid sequence that is at least 80% identical to an amino acid sequence selected from the group consisting of SEQ ID NOs: 1, 3, 5, 7, 9, 11, 13, 15, and 17; (c) transforming each of the first and the second genetically modified haploid yeast cells with one or more plasmids encoding a protein capable of complementing the endogenous mating impairment of said first and second genetically modified haploid yeast cells; (d) mating the first genetically modified haploid yeast cell with the second genetically modified haploid yeast cell, thereby forming a genetically modified diploid yeast cell; and (e) eliminating the one or more plasmids from the genetically modified diploid yeast cell, wherein the resulting genetically modified diploid yeast cell is sporulation and endogenous mating impaired and comprises two copies of said one or more heterologous nucleotide sequences encoding said one or more enzymes of the MEV pathway and two copies of said one or more nucleotide sequences encoding said one or more polypeptides having an amino acid sequence that is at least 80% identical to an amino acid sequence selected from the group consisting of SEQ ID NOs: 1, 3, 5, 7, 9, 11, 13, 15, and 17.

In some embodiments, the first genetically modified haploid yeast cell and the second genetically modified haploid yeast cell are endogenous mating impaired due to a functional disruption of one or more pheromone response genes. In some embodiments, step (c) of the method of the invention comprises transforming each of the first and the second genetically modified haploid yeast cells with one or more plasmids encoding a functional copy of the one or more pheromone response genes that are functionally disrupted in said first and second genetically modified haploid yeast cells. In some embodiments, the first and second genetically modified haploid yeast cells are haploid yeast cells and the one or more pheromone response genes are selected from the group consisting of STE5, STE4, STE18, STE12, STE7, and STE11. In certain embodiments, the first and second genetically modified haploid yeast cells are haploid yeast cells that are endogenous mating impaired due to a functional disruption of the STE5 gene.

In some embodiments, the first genetically modified haploid yeast cell and the second genetically modified haploid yeast cell are sporulation impaired due to a functional disruption of one or more sporulation genes. In some embodiments, the first and second genetically modified haploid yeast cells are haploid yeast cells and the one or more sporulation genes are selected from the group consisting of IME1, IME2, NDT80, SPO11, SPO20, AMA1, HOP2, and SPO21. In some embodiments, the first and second genetically modified haploid yeast cells are haploid yeast cells that are sporulation impaired due to a functional disruption of the IME1 gene. In particular embodiments, the first and second genetically modified haploid yeast cells are haploid yeast cells that are endogenous mating impaired due to a functional disruption of the STE5 gene, and are sporulation impaired due to a functional disruption of the IME1 gene.

In some embodiments, the second genetically modified haploid yeast cell is obtained by inducing a mating type switch in a population of the first genetically modified haploid yeast cell. In some embodiments, the first genetically modified haploid yeast cell is a heterothallic (ho) haploid Saccharomyces cerevisiae cell, and said population of heterothallic (ho) haploid Saccharomyces cerevisiae cell is induced to switch mating type by transforming said heterothallic (ho) haploid Saccharomyces cerevisiae cell with a plasmid encoding a homothallism (HO) protein, wherein expression of the HO protein induces a mating type switch in the haploid Saccharomyces cerevisiae cell to yield the second genetically modified haploid Saccharomyces cerevisiae cell. Heterothallic (ho) haploid Saccharomyces cerevisiae cells are characterized by the virtual non-occurrence of spontaneous mating type switching (frequency of only 10⁻⁶). By transiently expressing the HO protein, the frequency of spontaneous mating type switching in a haploid Saccharomyces cerevisiae cell can be increased to as much as once every cell division, providing a population of haploid cells of opposite mating types that can mate with each other to yield diploid Saccharomyces cerevisiae cells.

In other embodiments, the second genetically modified haploid yeast cell is obtained by changing the mating type locus in the first genetically modified haploid yeast cell using recombinant DNA technology. In some embodiments, the first genetically modified haploid yeast cell is transformed with an integration construct that comprises as an integrating sequence a nucleotide sequence that encodes a mating type other than the mating type of the first genetically modified haploid yeast cell, flanked by homologous sequences that are homologous to nucleotide sequences that flank the mating type locus in the first genetically modified haploid yeast cell. Upon integration of the integrating sequence via homologous recombination the mating type locus of the first genetically modified haploid yeast cell is replaced by the mating type locus encoded by the inserting sequence, resulting in the generation of the second genetically modified haploid yeast cell. In some embodiments, the integration construct is used to switch the mating type of the first genetically modified haploid yeast cell from a to alpha using an integration construct encoding encoding the alpha mating type (MAT alpha). In some embodiments, the integration construct comprises SEQ ID NO: 19. In other embodiments, the integration construct is used to switch the mating type of the first genetically modified haploid yeast cell from alpha to a using an integration construct encoding encoding the a mating type (MAT A). In some embodiments, the integration construct comprises SEQ ID NO: 20.

In another aspect, provided herein is a method for generating a genetically modified heterothallic (ho) diploid yeast cell that lacks sporulation and endogenous mating capability, the method comprising: (a) obtaining a first genetically modified heterothallic haploid yeast cell, wherein the first genetically modified heterothallic haploid yeast cell is sporulation and endogenous mating impaired and comprises one or more heterologous nucleotide sequences encoding one or more enzymes of the MEV pathway and one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17; (b) transforming the first genetically modified heterothallic haploid yeast cell with a plasmid encoding a homothallism (HO) protein to yield a first genetically modified haploid yeast cell, wherein expression of the HO protein induces a mating-type switch in the first genetically modified haploid yeast cell, whereby a second genetically modified haploid yeast cell is obtained, wherein the second genetically modified haploid yeast cell is sporulation and endogenous mating impaired, is of the opposite mating type as the first genetically modified haploid yeast cell, and comprises one or more heterologous nucleotide sequences encoding said one or more enzymes of the MEV pathway and said one or more nucleotide sequences encoding said one or more polypeptides having an amino acid sequence that is at least 80% identical to an amino acid sequence selected from the group consisting of SEQ ID NOs: 1, 3, 5, 7, 9, 11, 13, 15, and 17; (c) transforming each of the first and the second genetically modified haploid yeast cells with a plasmid encoding the one or more pheromone response proteins that are functionally disrupted in said first and second haploid yeast cell; (d) mating the first genetically modified haploid yeast cell with the second genetically modified haploid yeast cell, thereby forming a genetically modified diploid yeast cell that is homozygous other than for its mating type allele; and (e) eliminating any plasmids from the genetically modified diploid yeast cell to yield a genetically modified heterothallic diploid yeast cell, wherein the resulting genetically modified heterothallic diploid yeast cell is sporulation and endogenous mating impaired and comprises two copies of said one or more heterologous nucleotide sequences encoding said one or more enzymes of the MEV pathway and two copies of said one or more nucleotide sequences encoding said one or more polypeptides having an amino acid sequence that is at least 80% identical to an amino acid sequence selected from the group consisting of SEQ ID NOs: 1, 3, 5, 7, 9, 11, 13, 15, and 17.

Although the steps of the methods provided herein and described in greater detail below are presented in sequential order, one of skill in the art will recognize that the order of several steps can be interchanged, combined, or repeated without exceeding the scope of the invention. Thus, in some embodiments, a genetically modified heterothallic (ho) diploid yeast cell that lacks sporulation and endogenous mating capability is generated by first transforming a genetically modified heterothallic haploid yeast cell with a plasmid encoding one or more pheromone response proteins that are functionally disrupted in said genetically modified heterothallic haploid yeast cell, and then transforming the cell with a plasmid encoding a homothallism (HO) protein. In other embodiments, the genetically modified heterothallic (ho) diploid yeast cell that lacks sporulation and endogenous mating capability is generated by simultaneously transforming a genetically modified heterothallic haploid yeast cell with a plasmid encoding one or more pheromone response proteins that are functionally disrupted in said genetically modified heterothallic haploid yeast cell, and a plasmid encoding a homothallism (HO) protein.

In another aspect, provided herein is a method for producing an isoprenoid compound comprising: (a) obtaining a plurality of genetically modified yeast cells comprising one or more heterologous nucleotide sequences encoding one or more enzymes of the MEV pathway, and one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17; (b) culturing said genetically modified yeast cells in a medium comprising a carbon source under conditions suitable for making the isoprenoid compound; and (c) recovering the isoprenoid compound from the medium.

In some embodiments, the isoprenoid compound is a C₅ isoprenoid. These compounds are derived from one isoprene unit and are also called hemiterpenes. An illustrative example of a hemiterpene is isoprene. In other embodiments, the isoprenoid compound is a C₁₀ isoprenoid. These compounds are derived from two isoprene units and are also called monoterpenes. Illustrative examples of monoterpenes are limonene, citranellol, geraniol, menthol, perillyl alcohol, linalool, thujone, and myrcene. In other embodiments, the isoprenoid compound is a C₁₅ isoprenoid. These compounds are derived from three isoprene units and are also called sesquiterpenes. Illustrative examples of sesquiterpenes are periplanone B, gingkolide B, amorphadiene, artemisinin, artemisinic acid, valencene, nootkatone, epi-cedrol, epi-aristolochene, farnesol, gossypol, sanonin, periplanone, forskolin, and patchoulol (which is also known as patchouli alcohol). In other embodiments, the isoprenoid compound is a C₂₀ isoprenoid. These compounds are derived from four isoprene units and also called diterpenes. Illustrative examples of diterpenes are casbene, eleutherobin, paclitaxel, prostratin, pseudopterosin, and taxadiene. In yet other examples, the isoprenoid compound is a C₂₀₊ isoprenoid. These compounds are derived from more than four isoprene units and include: triterpenes (C₃₀ isoprenoid compounds derived from 6 isoprene units) such as arbrusideE, bruceantin, testosterone, progesterone, cortisone, digitoxin, and squalene; tetraterpenes (C₄₀ isoprenoid compounds derived from 8 isoprenoids) such as β-carotene; and polyterpenes (C₄₀₊ isoprenoid compounds derived from more than 8 isoprene units) such as polyisoprene. In some embodiments, the isoprenoid compound is selected from the group consisting of abietadiene, amorphadiene, carene, α-farnesene, β-farnesene, farnesol, geraniol, geranylgeraniol, isoprene, linalool, limonene, myrcene, nerolidol, ocimene, patchoulol, β-pinene, sabinene, γ-terpinene, terpinolene and valencene. Isoprenoid compounds also include, but are not limited to, carotenoids (such as lycopene, α- and β-carotene, α- and β-cryptoxanthin, bixin, zeaxanthin, astaxanthin, and lutein), steroid compounds, and compounds that are composed of isoprenoids modified by other chemical groups, such as mixed terpene-alkaloids, and coenzyme Q-10.

In some embodiments, the isoprenoid compound is produced in an amount greater than about 10 grams per liter of fermentation medium. In some embodiments, the isoprenoid compound is produced in an amount from about 10 to about 50 grams, more than about 15 grams, more than about 20 grams, more than about 25 grams, or more than about 30 grams per liter of cell culture.

In some embodiments, the isoprenoid compound is produced in an amount greater than about 50 milligrams per gram of dry cell weight. In some embodiments, the isoprenoid compound is produced in an amount from about 50 to about 1500 milligrams, more than about 100 milligrams, more than about 150 milligrams, more than about 200 milligrams, more than about 250 milligrams, more than about 500 milligrams, more than about 750 milligrams, or more than about 1000 milligrams per gram of dry cell weight.

In some embodiments, the isoprenoid compound produced in an amount that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the amount of the isoprenoid compound produced by a microbial cell that is not genetically modified according to the methods of the invention, on a per unit volume of cell culture basis.

In some embodiments, the isoprenoid compound produced in an amount that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the amount of the isoprenoid compound produced by a microbial cell that is not genetically modified according to the methods provided herein, on a per unit dry cell weight basis.

In some embodiments, the isoprenoid compound produced in an amount that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the amount of the isoprenoid compound produced by a microbial cell that is not genetically modified according to the methods provided herein, on a per unit volume of cell culture per unit time basis.

In some embodiments, the isoprenoid compound produced in an amount that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the amount of the isoprenoid compound produced by a microbial cell that is not genetically modified according to the methods provided herein, on a per unit dry cell weight per unit time basis.

In another aspect, provided herein is a method for detecting in a biological sample the presence or absence of a genetically modified microbial cell comprising one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17. In some embodiments, the method comprises: (a) obtaining a biological sample (e.g., a yeast cell and a population of yeast cells); (b) contacting the biological sample with a first compound or agent capable of interacting with a target molecule, wherein the target molecule is either a nucleic acid comprising a nucleotide sequence encoding a polypeptide having an amino acid sequence that is at least 80% identical to an amino acid sequence selected from the group consisting of SEQ ID NOs: 1, 3, 5, 7, 9, 11, 13, 15, and 17, or a polypeptide having an amino acid sequence that is at least 80% identical to an amino acid sequence selected from the group consisting of SEQ ID NOs: 1, 3, 5, 7, 9, 11, 13, 15, and 17; and (c) detecting said interaction of the first compound or agent with said target molecule, wherein detection of said interaction of the first compound or agent with the target molecule indicates the presence in the biological sample of a genetically modified microbial cell comprising one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17.

5.2.1 Microbe Selection

Microbes useful in the practice of the present invention include eukaryotic unicellular organisms, particularly fungi, and more particularly yeasts.

In some embodiments, yeasts useful in the present invention include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the microbe is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorpha (now known as Pichia angusta). In some embodiments, the microbe is a strain of the genus Candida, such as Candida lipolytica, Candida guilliermondii, Candida krusei, Candida pseudotropicalis, or Candida utilis.

In a particular embodiment, the microbe is Saccharomyces cerevisiae. In some embodiments, the microbe is a strain of Saccharomyces cerevisiae selected from the group consisting of Baker's yeast, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the microbe is a strain of Saccharomyces cerevisiae selected from the group consisting of PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

In some embodiments, the microbe is a microbe that is suitable for industrial fermentation, e.g., bioethanol fermentation. In particular embodiments, the microbe is conditioned to subsist under high solvent concentration, high temperature, expanded substrate utilization, nutrient limitation, osmotic stress due, acidity, sulfite and bacterial contamination, or combinations thereof, which are recognized stress conditions of the industrial fermentation environment.

5.2.2 Heterologous Nucleotide Sequences Encoding Enzymes of the MEV Pathway

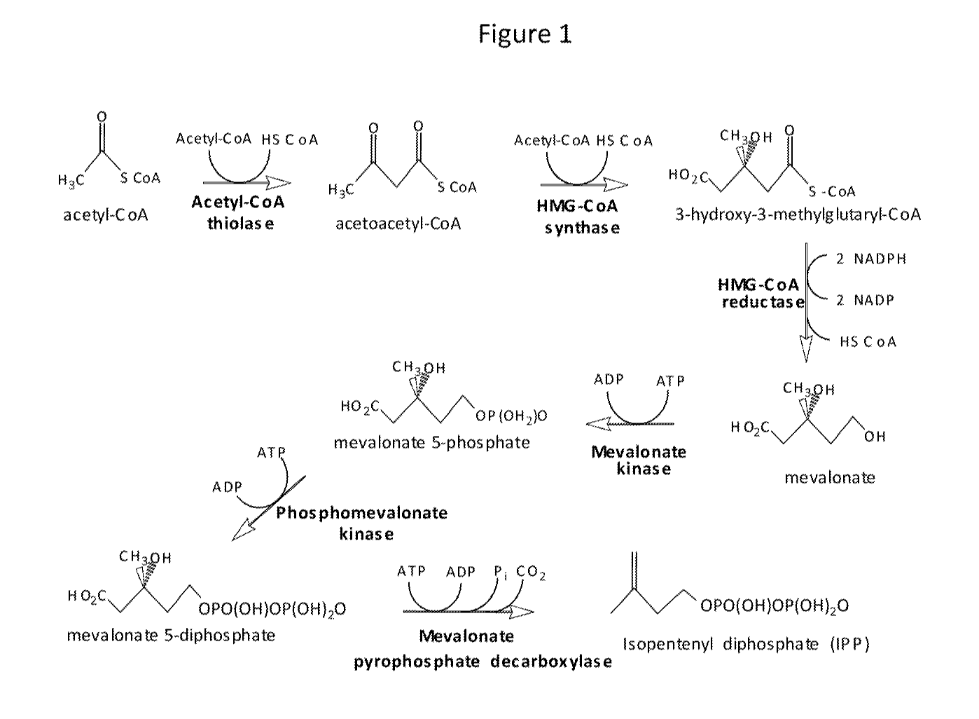

The genetically modified microbial cells of the invention comprise one or more heterologous nucleotide sequences encoding one or more MEV pathway enzymes to effect increased production of one or more isoprenoid compounds as compared to genetically unmodified parent microbial cells. Isoprenoids are derived from IPP, which can be biosynthesized by enzymes of the MEV pathway. A schematic representation of the MEV pathway is described in FIG. 1.

In some embodiments, the genetically modified microbial cell of the invention comprises a heterologous nucleotide sequence encoding an enzyme that can condense two molecules of acetyl-coenzyme A to form acetoacetyl-CoA, e.g., an acetyl-CoA thiolase. Illustrative examples of nucleotide sequences encoding such an enzyme include, but are not limited to: (NC_{—}000913 REGION: 2324131 . . . 2325315; Escherichia coli), (D49362; Paracoccus denitrificans), and (L20428; Saccharomyces cerevisiae).

In some embodiments, the genetically modified microbial cell of the invention comprises a heterologous nucleotide sequence encoding an enzyme that can condense acetoacetyl-CoA with another molecule of acetyl-CoA to form 3-hydroxy-3-methylglutaryl-CoA (HMG-CoA), e.g., a HMG-CoA synthase. Illustrative examples of nucleotide sequences encoding such an enzyme include, but are not limited to: (NC_{—}001145. complement 19061 . . . 20536; Saccharomyces cerevisiae), (X96617; Saccharomyces cerevisiae), (X83882; Arabidopsis thaliana), (AB037907; Kitasatospora griseola), (BT007302; Homo sapiens), and (NC_{—}002758, Locus tag SAV2546, GeneID 1122571; Staphylococcus aureus).

In some embodiments, the genetically modified microbial cell of the invention comprises a heterologous nucleotide sequence encoding an enzyme that can convert HMG-CoA into mevalonate, e.g., a HMG-CoA reductase. Illustrative examples of nucleotide sequences encoding such an enzyme include, but are not limited to: (NM_{—}206548; Drosophila melanogaster), (NC_{—}002758, Locus tag SAV2545, GeneID 1122570; Staphylococcus aureus), (NM_{—}204485; Gallus gallus), (AB015627; Streptomyces sp. KO 3988), (AF542543; Nicotiana attenuata), (AB037907; Kitasatospora griseola), (AX 128213, providing the sequence encoding a truncated HMGR; Saccharomyces cerevisiae), and (NC_{—}001145: complement (115734 . . . 118898; Saccharomyces cerevisiae).

In some embodiments, the genetically modified microbial cell of the invention comprises a heterologous nucleotide sequence encoding an enzyme that can convert mevalonate into mevalonate 5-phosphate, e.g., a mevalonate kinase. Illustrative examples of nucleotide sequences encoding such an enzyme include, but are not limited to: (L77688; Arabidopsis thaliana), and (X55875; Saccharomyces cerevisiae).

In some embodiments, the genetically modified microbial cell of the invention comprises a heterologous nucleotide sequence encoding an enzyme that can convert mevalonate 5-phosphate into mevalonate 5-pyrophosphate, e.g., a phosphomevalonate kinase. Illustrative examples of nucleotide sequences encoding such an enzyme include, but are not limited to: (AF429385; Hevea brasiliensis), (NM_{—}006556; Homo sapiens), and (NC_{—}001145. complement 712315 . . . 713670; Saccharomyces cerevisiae).

In some embodiments, the genetically modified microbial cell of the invention comprises a heterologous nucleotide sequence encoding an enzyme that can convert mevalonate 5-pyrophosphate into IPP, e.g., a mevalonate pyrophosphate decarboxylase. Illustrative examples of nucleotide sequences encoding such an enzyme include, but are not limited to: (X97557; Saccharomyces cerevisiae), (AF290095; Enterococcus faecium), and (U49260; Homo sapiens).

5.2.3 PE-2 Nucleotide Sequences

The genetically modified microbial cell of the invention comprises one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17.

In some embodiments, the polypeptide has an amino acid sequence that is at least 80% identical to SEQ ID NO: 1. In some embodiments, the polypeptide has an amino acid sequence that is at least 80% identical to SEQ ID NO: 3. In some embodiments, the polypeptide has an amino acid sequence that is at least 80% identical to SEQ ID NO: 5. In some embodiments, the polypeptide has an amino acid sequence that is at least 80% identical to SEQ ID NO: 7. In some embodiments, the polypeptide has an amino acid sequence that is at least 80% identical to SEQ ID NO: 9. In some embodiments, the polypeptide has an amino acid sequence that is at least 80% identical to SEQ ID NO: 11. In some embodiments, the polypeptide has an amino acid sequence that is at least 80% identical to SEQ ID NO: 13. In some embodiments, the polypeptide has an amino acid sequence that is at least 80% identical to SEQ ID NO: 15. In some embodiments, the polypeptide has an amino acid sequence that is at least 80% identical to SEQ ID NO: 17.

In some embodiments, the polypeptide has an amino acid sequence that is SEQ ID NO: 1. In some embodiments, the polypeptide has an amino acid sequence that is SEQ ID NO: 3. In some embodiments, the polypeptide has an amino acid sequence that is SEQ ID NO: 5. In some embodiments, the polypeptide has an amino acid sequence that is SEQ ID NO: 7. In some embodiments, the polypeptide has an amino acid sequence that is SEQ ID NO: 9. In some embodiments, the polypeptide has an amino acid sequence that is SEQ ID NO: 11. In some embodiments, the polypeptide has an amino acid sequence that is SEQ ID NO: 13. In some embodiments, the polypeptide has an amino acid sequence that is SEQ ID NO: 15. In some embodiments, the polypeptide has an amino acid sequence that is SEQ ID NO: 17.

In some embodiments the nucleotide sequence is at least 85% identical to SEQ ID NO: 2 or to the complement thereof. In some embodiments the nucleotide sequence is at least 85% identical to SEQ ID NO: 4 or to the complement thereof. In some embodiments the nucleotide sequence is at least 85% identical to SEQ ID NO: 6 or to the complement thereof. In some embodiments the nucleotide sequence is at least 85% identical to SEQ ID NO: 8 or to the complement thereof. In some embodiments the nucleotide sequence is at least 85% identical to SEQ ID NO: 10 or to the complement thereof. In some embodiments the nucleotide sequence is at least 85% identical to SEQ ID NO: 12 or to the complement thereof. In some embodiments the nucleotide sequence is at least 85% identical to SEQ ID NO: 14 or to the complement thereof. In some embodiments the nucleotide sequence is at least 85% identical to SEQ ID NO: 16 or to the complement thereof. In some embodiments the nucleotide sequence is at least 85% identical to SEQ ID NO: 18 or to the complement thereof.

In some embodiments the nucleotide sequence is SEQ ID NO: 2 or the complement thereof. In some embodiments the nucleotide sequence is SEQ ID NO: 4 or the complement thereof. In some embodiments the nucleotide sequence is SEQ ID NO: 6 or the complement thereof. In some embodiments the nucleotide sequence is SEQ ID NO: 8 or the complement thereof. In some embodiments the nucleotide sequence is SEQ ID NO: 10 or the complement thereof. In some embodiments the nucleotide sequence is SEQ ID NO: 12 or the complement thereof. In some embodiments the nucleotide sequence is SEQ ID NO: 14 or the complement thereof. In some embodiments the nucleotide sequence is SEQ ID NO: 16 or the complement thereof. In some embodiments the nucleotide sequence is SEQ ID NO: 18 or the complement thereof.

Percent identity in this context means the percentage of amino acid residues or nucleotides in the candidate sequence that are identical (i.e., the amino acid residues or nucleotides at given positions in the alignment are the same) or similar (i.e., the amino acid residue at a given position in the alignment is substituted with a different amino acids such that the substitution has no material effect on the biological activity of the polypeptide (conservative substitution), e.g., substitution of one basic residue for another (e.g., Arg for Lys), substitution of one hydrophobic residue for another (e.g., Leu for Ile), or substitution of one aromatic residue for another (e.g., Phe for Tyr)) after aligning the sequences and introducing gaps, if necessary, to achieve the maximum percent sequence homology. Amino acids for which conservative substitutions can be made are well known in the art. Amino acid substitutions can be made by changing the nucleotide sequence encoding the polypeptide. Changes in nucleotide sequence can be made using methods known in the art, such as oligonucleotide-mediated (site-directed) mutagenesis (see Carter, Biochem. J. 237:1-7 (1986); Zoller and Smith, Methods Enzymol. 154:329-50 (1987)), PCR mutagenesis, cassette mutagenesis, restriction selection mutagenesis (Wells et al., Gene 34:315-323 (1985)), or other known techniques. See, for example, Ausubel et al., Current Protocols In Molecular Biology, John Wiley and Sons, New York (current edition); and Sambrook et al., Molecular Cloning, A Laboratory Manual, 3d. ed., Cold Spring Harbor Laboratory Press, Cold Spring Harbor, N.Y. (2001).

Percent identity is determined using sequence alignment techniques and computer algorithms well-known in the art, preferably integrated in software packages designed for this purpose, using the default parameters of said computer algorithms or software packages. Non-limiting examples of suitable computer algorithms and software packages include the following: the BLAST family of programs (e.g., Karlin & Altschul, 1990, Proc. Natl. Acad. Sci. USA 87:2264-2268 (modified as in Karlin & Altschul, 1993, Proc. Natl. Acad. Sci. USA 90:5873-5877), Altschul et al., 1990, J. Mol. Biol. 215:403-410, (describing NBLAST and XBLAST), Altschul et al., 1997, Nucleic Acids Res. 25:3389-3402 (describing Gapped BLAST, and PSI-Blast) (for pairwise DNA-DNA comparison, the BLASTN 2.1.2 program can be used with default parameters (Match: 1; Mismatch: −2; Open gap: 5 penalties; extension gap: 2 penalties; gap x_dropoff: 50; expect: 10; and word size: 11, with filter); for pairwise protein-protein sequence comparison, the BLASTP 2.1.2 program can be employed using default parameters (Matrix: BLOSUM62; gap open: 11; gap extension: 1; x_dropoff: 15; expect: 10.0; and wordsize: 3, with filter)); the algorithm of Myers and Miller, 1989, Math. Biol., 51:5-37, and 1988, Comput. Appl. Biosci., 4(1):11-17, which is incorporated into the ALIGN program (version 2.0) and is available as part of the GCG sequence alignment software package and which is suitable when the two sequences being compared are dissimar in length; the FASTA program (Pearson W. R. and Lipman D. J., Proc. Nat. Acad. Sci. USA, 85:2444-2448, 1988), available as part of the Wisconsin Sequence Analysis Package; BESTFIT, which uses the “local homology” algorithm of Smith and Waterman (Advances in Applied Mathematics, 2:482-489, 1981) to find best single region of similarity between two sequences, and which is preferable where the two sequences being compared are dissimilar in length; and GAP, which aligns two sequences by finding a “maximum similarity” according to the algorithm of Neddleman and Wunsch (J. Mol. Biol. 48:443-354, 1970), and is preferable where the two sequences are approximately the same length and an alignment is expected over the entire length.

5.2.4 Further Genetic Modifications

5.2.4.1 IPP to Isoprenoids

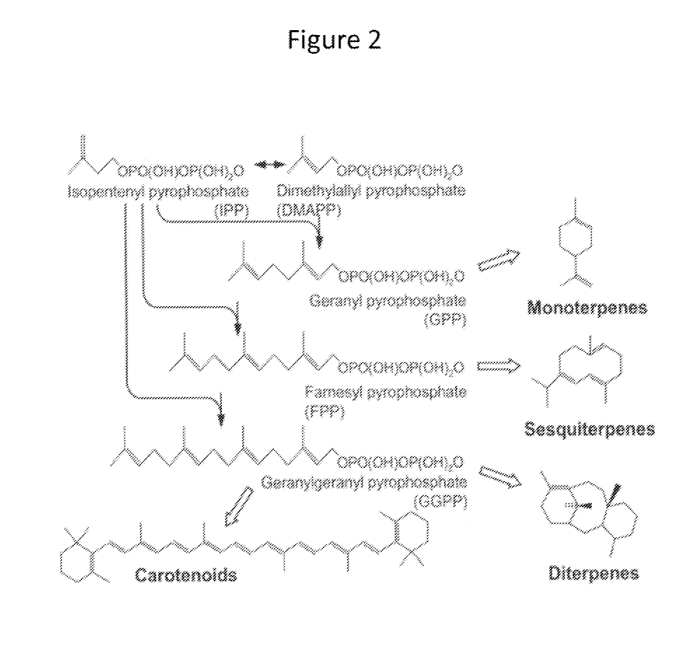

IPP generated via the MEV pathway can be converted to its isomer, DMAPP, condensed, and modified through the action of various additional enzymes to form simple and more complex isoprenoids (FIG. 2).

In some embodiments, the genetically modified microbial cell of the invention further comprises a heterologous nucleotide sequence encoding an enzyme that can convert IPP generated via the MEV pathway into DMAPP, e.g., an IPP isomerase. Illustrative examples of nucleotide sequences encoding such an enzyme include, but are not limited to: (NC_{—}000913, 3031087 . . . 3031635; Escherichia coli), and (AF082326; Haematococcus pluvialis).

In some embodiments, the genetically modified microbial cell of the invention further comprises a heterologous nucleotide sequence encoding a polyprenyl synthase that can condense IPP and/or DMAPP molecules to form polyprenyl compounds containing more than five carbons.

In some embodiments, the genetically modified microbial cell of the invention comprises a heterologous nucleotide sequence encoding an enzyme that can condense one molecule of IPP with one molecule of DMAPP to form one molecule of GPP, e.g., a GPP synthase. Illustrative examples of nucleotide sequences encoding such an enzyme include, but are not limited to: (AF513111; Abies grandis), (AF513112; Abies grandis), (AF513113; Abies grandis), (AY534686; Antirrhinum majus), (AY534687; Antirrhinum majus), (Y17376; Arabidopsis thaliana), (AE016877, Locus AP11092; Bacillus cereus; ATCC 14579), (AJ243739; Citrus sinensis), (AY534745; Clarkia breweri), (AY953508; Ips pini), (DQ286930; Lycopersicon esculentum), (AF182828; Mentha×piperita), (AF182827; Mentha×piperita), (MP1249453; Mentha×piperita), (PZE431697, Locus CAD24425; Paracoccus zeaxanthinifaciens), (AY866498; Picrorhiza kurrooa), (AY351862; Vitis vinifera), and (AF2038891, Locus AAF12843; Zymomonas mobilis).

In some embodiments, the genetically modified microbial cell of the invention comprises a heterologous nucleotide sequence encoding an enzyme that can condense two molecules of IPP with one molecule of DMAPP, or add a molecule of IPP to a molecule of GPP, to form a molecule of FPP, e.g., a FPP synthase. Illustrative examples of nucleotide sequences that encode such an enzyme include, but are not limited to: (ATU80605; Arabidopsis thaliana), (ATHFPS2R; Arabidopsis thaliana), (AAU36376; Artemisia annua), (AF461050; Bos taurus), (D00694; Escherichia coli K-12), (AE009951, Locus AAL95523; Fusobacterium nucleatum subsp. nucleatum ATCC 25586), (GFFPPSGEN; Gibberella fujikuroi), (CP000009, Locus AAW60034; Gluconobacter oxydans 621H), (AF019892; Helianthus annuus), (HUMFAPS; Homo sapiens), (KLPFPSQCR; Kluyveromyces lactis), (LAU15777; Lupinus albus), (LAU20771; Lupinus albus), (AF309508; Mus musculus), (NCFPPSGEN; Neurospora crassa), (PAFPS1; Parthenium argentatum), (PAFPS2; Parthenium argentatum), (RATFAPS; Rattus norvegicus), (YSCFPP; Saccharomyces cerevisiae), (D89104; Schizosaccharomyces pombe), (CP000003, Locus AAT87386; Streptococcus pyogenes), (CP000017, Locus AAZ51849; Streptococcus pyogenes), (NC_{—}008022, Locus YP_{—}598856; Streptococcus pyogenes MGAS10270), (NC_{—}008023, Locus YP_{—}600845; Streptococcus pyogenes MGAS2096), (NC_{—}008024, Locus YP_{—}602832; Streptococcus pyogenes MGAS10750), (MZEFPS; Zea mays), (AE000657, Locus AAC06913; Aquifex aeolicus VF5), (NM_{—}202836; Arabidopsis thaliana), (D84432, Locus BAA 12575; Bacillus subtilis), (U12678, Locus AAC28894; Bradyrhizobium japonicum USDA 110), (BACFDPS; Geobacillus stearothermophilus), (NC_{—}002940, Locus NP_{—}873754; Haemophilus ducreyi 35000HP), (L42023, Locus AAC23087; Haemophilus influenzae Rd KW20), (J05262; Homo sapiens), (YP_{—}395294; Lactobacillus sakei subsp. sakei 23K), (NC_{—}005823, Locus YP_{—}000273; Leptospira interrogans serovar Copenhageni str. Fiocruz L1-130), (AB003187; Micrococcus luteus), (NC_{—}002946, Locus YP_{—}208768; Neisseria gonorrhoeae FA 1090), (U00090, Locus AAB91752; Rhizobium sp. NGR234), (J05091; Saccharomyces cerevisae), (CP000031, Locus AAV93568; Silicibacter pomeroyi DSS-3), (AE008481, Locus AAK99890; Streptococcus pneumoniae R6), and (NC_{—}004556, Locus NP 779706; Xylella fastidiosa Temecula1).

In some embodiments, the genetically modified microbial cell of the invention further comprises a heterologous nucleotide sequence encoding an enzyme that can combine IPP and DMAPP or IPP and FPP to form GGPP. Illustrative examples of nucleotide sequences that encode such an enzyme include, but are not limited to: (ATHGERPYRS; Arabidopsis thaliana), (BT005328; Arabidopsis thaliana), (NM_{—}119845; Arabidopsis thaliana), (NZ_AAJM01000380, Locus ZP_{—}00743052; Bacillus thuringiensis serovar israelensis, ATCC 35646 sq1563), (CRGGPPS; Catharanthus roseus), (NZ_AABF02000074, Locus ZP_{—}00144509; Fusobacterium nucleatum subsp. vincentii, ATCC 49256), (GFGGPPSGN; Gibberella fujikuroi), (AY371321; Ginkgo biloba), (AB055496; Hevea brasiliensis), (AB017971; Homo sapiens), (MCI276129; Mucor circinelloides f. lusitanicus), (AB016044; Mus musculus), (AABX01000298, Locus NCU01427; Neurospora crassa), (NCU20940; Neurospora crassa), (NZ_AAKL01000008, Locus ZP_{—}00943566; Ralstonia solanacearum UW551), (AB118238; Rattus norvegicus), (SCU31632; Saccharomyces cerevisiae), (AB016095; Synechococcus elongates), (SAGGPS; Sinapis alba), (SSOGDS; Sulfolobus acidocaldarius), (NC_{—}007759, Locus YP_{—}461832; Syntrophus aciditrophicus SB), (NC_{—}006840, Locus YP_{—}204095; Vibrio fischeri ES 114), (NM_{—}112315; Arabidopsis thaliana), (ERWCRTE; Pantoea agglomerans), (D90087, Locus BAA14124; Pantoea ananatis), (X52291, Locus CAA36538; Rhodobacter capsulatus), (AF195122, Locus AAF24294; Rhodobacter sphaeroides), and (NC_{—}004350, Locus NP_{—}721015; Streptococcus mutans UA159).

In some embodiments, the genetically modified microbial cell of the invention further comprises a heterologous nucleotide sequence encoding an enzyme that can modify a polyprenyl to form a hemiterpene, a monoterpene, a sesquiterpene, a diterpene, a triterpene, a tetraterpene, a polyterpene, a steroid compound, a carotenoid, or a modified isoprenoid compound.

In some embodiments, the heterologous nucleotide encodes a carene synthase. Illustrative examples of suitable nucleotide sequences include, but are not limited to: (AF461460, REGION 43 . . . 1926; Picea abies) and (AF527416, REGION: 78 . . . 1871; Salvia stenophylla).

In some embodiments, the heterologous nucleotide encodes a geraniol synthase. Illustrative examples of suitable nucleotide sequences include, but are not limited to: (AJ457070; Cinnamomum tenuipilum), (AY362553; Ocimum basilicum), (DQ234300; Perilla frutescens strain 1864), (DQ234299; Perilla citriodora strain 1861), (DQ234298; Perilla citriodora strain 4935), and (DQ088667; Perilla citriodora).

In some embodiments, the heterologous nucleotide encodes a linalool synthase. Illustrative examples of a suitable nucleotide sequence include, but are not limited to: (AF497485; Arabidopsis thaliana), (AC002294, Locus AAB71482; Arabidopsis thaliana), (AY059757; Arabidopsis thaliana), (NM_{—}104793; Arabidopsis thaliana), (AF154124; Artemisia annua), (AF067603; Clarkia breweri), (AF067602; Clarkia concinna), (AF067601; Clarkia breweri), (U58314; Clarkia breweri); (AY840091; Lycopersicon esculentum), (DQ263741; Lavandula angustifolia), (AY083653; Mentha citrate), (AY693647; Ocimum basilicum), (XM_{—}463918; Oryza sativa), (AP004078, Locus BAD07605; Oryza sativa), (XM_{—}463918, Locus XP_{—}463918; Oryza sativa), (AY917193; Perilla citriodora), (AF271259; Perilla frutescens), (AY473623; Picea abies), (DQ195274; Picea sitchensis), and (AF444798; Perilla frutescens var. crispa cultivar No. 79).

In some embodiments, the heterologous nucleotide encodes a limonene synthase. Illustrative examples of suitable nucleotide sequences include, but are not limited to: (+)-limonene synthases (AF514287, REGION: 47 . . . 1867; Citrus limon) and (AY055214, REGION: 48 . . . 1889; Agastache rugosa) and (−)-limonene synthases (DQ195275, REGION: 1 . . . 1905; Picea sitchensis), (AF006193, REGION: 73 . . . 1986; Abies grandis), and (MHC4SLSP, REGION: 29 . . . 1828; Mentha spicata).

In some embodiments, the heterologous nucleotide encodes a myrcene synthase. Illustrative examples of suitable nucleotide sequences include, but are not limited to: (U87908; Abies grandis), (AY195609; Antirrhinum majus), (AY195608; Antirrhinum majus), (NM_{—}127982; Arabidopsis thaliana TPS10), (NM_{—}113485; Arabidopsis thaliana ATTPS-CIN), (NM_{—}113483; Arabidopsis thaliana ATTPS-CIN), (AF271259; Perilla frutescens), (AY473626; Picea abies), (AF369919; Picea abies), and (AJ304839; Quercus ilex).

In some embodiments, the heterologous nucleotide encodes a ocimene synthase. Illustrative examples of suitable nucleotide sequences include, but are not limited to: (AY195607; Antirrhinum majus), (AY195609; Antirrhinum majus), (AY195608; Antirrhinum majus), (AK221024; Arabidopsis thaliana), (NM_{—}113485; Arabidopsis thaliana ATTPS-CIN), (NM_{—}113483; Arabidopsis thaliana ATTPS-CIN), (NM_{—}117775; Arabidopsis thaliana ATTPS03), (NM_{—}001036574; Arabidopsis thaliana ATTPS03), (NM_{—}127982; Arabidopsis thaliana TPS10), (AB110642; Citrus unshiu CitMTSL4), and (AY575970; Lotus corniculatus var. japonicus).

In some embodiments, the heterologous nucleotide encodes an α-pinene synthase. Illustrative examples of suitable nucleotide sequences include, but are not limited to: (+) α-pinene synthase (AF543530, REGION: 1 . . . 1887; Pinus taeda), (−)α-pinene synthase (AF543527, REGION: 32 . . . 1921; Pinus taeda), and (+)/(−)α-pinene synthase (AGU87909, REGION: 6111892; Abies grandis).

In some embodiments, the heterologous nucleotide encodes a β-pinene synthase. Illustrative examples of suitable nucleotide sequences include, but are not limited to: (−) β-pinene synthases (AF276072, REGION: 1 . . . 1749; Artemisia annua) and (AF514288, REGION: 26 . . . 1834; Citrus limon).

In some embodiments, the heterologous nucleotide encodes a sabinene synthase. An illustrative example of a suitable nucleotide sequence includes but is not limited to AF051901, REGION: 26 . . . 1798 from Salvia officinalis.

In some embodiments, the heterologous nucleotide encodes a γ-terpinene synthase. Illustrative examples of suitable nucleotide sequences include: (AF514286, REGION: 30 . . . 1832 from Citrus limon) and (AB110640, REGION 1 . . . 1803 from Citrus unshiu).

In some embodiments, the heterologous nucleotide encodes a terpinolene synthase. Illustrative examples of a suitable nucleotide sequence include but is not limited to: (AY693650 from Oscimum basilicum) and (AY906866, REGION: 10 . . . 1887 from Pseudotsuga menziesii).

In some embodiments, the heterologous nucleotide encodes a amorphadiene synthase. An illustrative example of a suitable nucleotide sequence is SEQ ID NO. 37 of U.S. Patent Publication No. 2004/0005678.

In some embodiments, the heterologous nucleotide encodes a α-farnesene synthase. Illustrative examples of suitable nucleotide sequences include, but are not limited to DQ309034 from Pyrus communis cultivar d'Anjou (pear; gene name AFS1) and AY182241 from Malus domestica (apple; gene AFS1). Pechouus et al., Planta 219(1):84-94 (2004).

In some embodiments, the heterologous nucleotide encodes a β-farnesene synthase. Illustrative examples of suitable nucleotide sequences include but is not limited to GenBank accession number AF024615 from Mentha×piperita (peppermint; gene Tspa11), and AY835398 from Artemisia annua. Picaud et al., Phytochemistry 66(9): 961-967 (2005).

In some embodiments, the heterologous nucleotide encodes a farnesol synthase. Illustrative examples of suitable nucleotide sequences include, but are not limited to GenBank accession number AF529266 from Zea mays and YDR481c from Saccharomyces cerevisiae (gene Pho8). Song, L., Applied Biochemistry and Biotechnology 128:149-158 (2006).

In some embodiments, the heterologous nucleotide encodes a nerolidol synthase. An illustrative example of a suitable nucleotide sequence includes but is not limited to AF529266 from Zea mays (maize; gene tps1).

In some embodiments, the heterologous nucleotide encodes a patchouliol synthase. Illustrative examples of suitable nucleotide sequences include, but are not limited to AY508730 REGION: 1 . . . 1659 from Pogostemon cablin.

In some embodiments, the heterologous nucleotide encodes a nootkatone synthase. Illustrative examples of a suitable nucleotide sequence includes but is not limited to AF441124 REGION: 1 . . . 1647 from Citrus sinensis and AY917195 REGION: 1 . . . 1653 from Perilla frutescens.

In some embodiments, the heterologous nucleotide encodes an abietadiene synthase. Illustrative examples of suitable nucleotide sequences include, but are not limited to: (U50768; Abies grandis) and (AY473621; Picea abies).

5.2.4.2 Flocculation

In certain embodiments, the genetically modified microbial cell of the invention comprises one or more heterologous nucleotide sequences encoding one or more flocculation proteins.

In some embodiments, the flocculation protein is Flo1p. Representative FLO1 nucleotide sequences of Saccharomyces cerevisiae include, but are not limited to Genbank accession numbers NM_{—}001178230, AY949848, and X78160. Representative Flo1p amino acid sequences of Saccharomyces cerevisiae include, but are not limited to Genbank accession numbers NP_{—}009424, AAX47297, and CAA55024.

In some embodiments, the flocculation protein is Flo1p. Representative FLO5 nucleotide sequences of Saccharomyces cerevisiae include, but are not limited to Genbank accession number NM_{—}00117934. Representative Flo5p amino acid sequences of Saccharomyces cerevisiae include, but are not limited to Genbank accession number NP_{—}012081.

In some embodiments, the flocculation protein is Flo8p. Representative FLO8 nucleotide sequences of Saccharomyces cerevisiae include, but are not limited to Genbank accession numbers YSCFL08 and NM_{—}001178999. Representative Flo8p amino acid sequences of Saccharomyces cerevisiae include, but are not limited to Genbank accession numbers BAA 12076 and NP_{—}011034.

In some embodiments, the flocculation protein is Flo9p. Representative FLO9 nucleotide sequences of Saccharomyces cerevisiae include, but are not limited to Genbank accession number NM_{—}001178205. Representative Flo9p amino acid sequences of Saccharomyces cerevisiae include, but are not limited to Genbank accession number NP_{—}009338.

In some embodiments, the flocculation protein is Flo10p. Representative FLO10 nucleotide sequences of Saccharomyces cerevisiae include, but are not limited to Genbank accession number NM_{—}001179892. Representative Flo10p amino acid sequences of Saccharomyces cerevisiae include, but are not limited to Genbank accession number NP_{—}013028.

In some embodiments, the flocculation protein is Flo11p. Representative FLO11 nucleotide sequences of Saccharomyces cerevisiae include, but are not limited to Genbank accession number NM_{—}001179541. Representative Flo11p amino acid sequences of Saccharomyces cerevisiae include, but are not limited to Genbank accession number NP_{—}012284.

5.2.4.3 Pheromone Response Genes

In some embodiments, the genetically modified yeast cell of the invention comprises a functional disruption in a pheromone response gene. In some embodiments, the pheromone response gene is STE5. Representative STE5 nucleotide sequences of Saccharomyces cerevisiae include Genbank accession number L23856 and sequences identified as SEQ ID NOS: 17, 45, 73, 101, and 129 in U.S. patent application Ser. No. ______ (entitled “Methods for Generating a Genetically Modified Microbe”; Attorney Docket No. 11836-045-999), filed Jun. 1, 2010. Representative Step 5p amino acid sequences of Saccharomyces cerevisiae include Genbank accession number AAA35115 and sequences identified as SEQ ID NOS: 18, 46, 74, 102, and 130 in U.S. patent application Ser. No. ______ (entitled “Methods for Generating a Genetically Modified Microbe”; Attorney Docket No. 11836-045-999), filed Jun. 1, 2010.

In some embodiments, the pheromone response gene is STE4. The sequence of the STE4 gene of Saccharomyces cerevisiae has been previously described. Dujon et al., Nature 387 (6632 Suppl):98-102 (1997). Representative STE4 nucleotide sequences of Saccharomyces cerevisiae include Genbank accession number NC_{—}001147.5 and sequences identified as SEQ ID NOS: 19, 47, 75, 103, and 131 in U.S. patent application Ser. No. ______ (entitled “Methods for Generating a Genetically Modified Microbe”; Attorney Docket No. 11836-045-999), filed Jun. 1, 2010. Representative Step 4p amino acid sequences of Saccharomyces cerevisiae include Genbank accession number NP_{—}014855 and sequences identified as SEQ ID NOS: 20, 48, 76, 104, and 132 in U.S. patent application Ser. No. ______ (entitled “Methods for Generating a Genetically Modified Microbe”; Attorney Docket No. 11836-045-999), filed Jun. 1, 2010.

In some embodiments, the pheromone response gene is STE18. The sequence of the STE18 gene of Saccharomyces cerevisiae has been previously described. See, e.g., Goffeau et al., Science 274 (5287):546-547 (1996). Representative STE18 nucleotide sequences of Saccharomyces cerevisiae include Genbank accession number NC_{—}001147.5 and sequences identified as SEQ ID NOS: 21, 49, 77, 105, and 133 in U.S. patent application Ser. No. ______ (entitled “Methods for Generating a Genetically Modified Microbe”; Attorney Docket No. 11836-045-999), filed Jun. 1, 2010. Representative Ste18p amino acid sequences of Saccharomyces cerevisiae include Genbank accession number NP_{—}012619 and sequences identified as SEQ ID NOS: 22, 50, 78, 106, and 134 in U.S. patent application Ser. No. ______ (entitled “Methods for Generating a Genetically Modified Microbe”; Attorney Docket No. 11836-045-999), filed Jun. 1, 2010.

In some embodiments, the pheromone response gene is STE12. The sequence of the STE12 gene of Saccharomyces cerevisiae has been previously described. See, e.g., Goffeau et al., Science 274 (5287):546-547 (1996). Representative STE12 nucleotide sequences of Saccharomyces cerevisiae include Genbank accession number NC_{—}001140.5 and sequences identified as SEQ ID NOS: 23, 51, 79, 107, and 135 in U.S. patent application Ser. No. ______ (entitled “Methods for Generating a Genetically Modified Microbe”; Attorney Docket No. 11836-045-999), filed Jun. 1, 2010. Representative Ste12p amino acid sequences of Saccharomyces cerevisiae include Genbank accession number NP_{—}011952 and sequences identified as SEQ ID NOS: 24, 52, 80, 108 and 136 in U.S. patent application Ser. No. ______ (entitled “Methods for Generating a Genetically Modified Microbe”; Attorney Docket No. 11836-045-999), filed Jun. 1, 2010.

In some embodiments, the pheromone response gene is STE7. The sequence of the STE7 gene of Saccharomyces cerevisiae has been previously described. See, e.g., Teague et al., Proc Natl Ac ad Sci USA. 83(19):7371-5 (1986). Representative STE7 nucleotide sequences of Saccharomyces cerevisiae include Genbank accession number Z74207 and sequences identified as SEQ ID NOS: 25, 53, 81, 109, and 137 in U.S. patent application Ser. No. ______ (entitled “Methods for Generating a Genetically Modified Microbe”; Attorney Docket No. 11836-045-999), filed Jun. 1, 2010. Representative Step 7p amino acid sequences of Saccharomyces cerevisiae include Genbank accession number CAA98732 and sequences identified as SEQ ID NOS: 26, 54, 82, 110, and 138 in U.S. patent application Ser. No. ______ (entitled “Methods for Generating a Genetically Modified Microbe”; Attorney Docket No. 11836-045-999), filed Jun. 1, 2010.

In some embodiments, the pheromone response gene is STE11. The sequence of the STE11 gene of Saccharomyces cerevisiae has been previously described. See, e.g., Johnston et al., Nature 387 (6632 Suppl), 87-90 (1997). Representative STE11 nucleotide sequences of Saccharomyces cerevisiae include Genbank accession number NC_{—}001144.4 and sequences identified as SEQ ID NOS: 27, 55, 83, 111, and 139 in U.S. patent application Ser. No. ______ (entitled “Methods for Generating a Genetically Modified Microbe”; Attorney Docket No. 11836-045-999), filed Jun. 1, 2010. Representative Ste11p amino acid sequences of Saccharomyces cerevisiae include Genbank accession number NP_{—}013466 and sequences identified as SEQ ID NOS: 28, 56, 84, 112, and 140 in U.S. patent application. Ser. No. ______ (entitled “Methods for Generating a Genetically Modified Microbe”; Attorney Docket No. 11836-045-999), filed Jun. 1, 2010.

5.2.4.4 Sporulation Genes

In some embodiments, the genetically modified yeast cell of the invention comprises a functional disruption in a sporulation gene.

In some embodiments, the sporulation gene is IME1. The sequence of the IME1 gene of Saccharomyces cerevisiae has been previously described. See, e.g., Smith, H. E., et al., Mol. Cell. Biol. 10 (12):6103-6113 (1990). Representative IME1 nucleotide sequences of Saccharomyces cerevisiae include Genbank accession number M37188 and sequences identified as SEQ ID NOS: 1, 29, 57, 85, and 113 in U.S. patent application Ser. No. ______ (entitled “Methods for Generating a Genetically Modified Microbe”; Attorney Docket No. 11836-045-999), filed Jun. 1, 2010. Representative Ime1p amino acid sequences of Saccharomyces cerevisiae include Genbank accession number AAA86790 and sequences identified as SEQ ID NOS: 2, 30, 58, 86, and 114 in U.S. patent application Ser. No. ______ (entitled “Methods for Generating a Genetically Modified Microbe”; Attorney Docket No. 11836-045-999), filed Jun. 1, 2010.

In some embodiments, the sporulation gene is IME2. The sequence of the IME2 gene of Saccharomyces cerevisiae has been previously described. See, e.g., EMBO J. (9), 2031-2049 (1996). Representative IME2 nucleotide sequences of Saccharomyces cerevisiae include Genbank accession number NC_{—}001142 and sequences identified as SEQ ID NOS: 3, 31, 59, 87, and 115 in U.S. patent application Ser. No. ______ (entitled “Methods for Generating a Genetically Modified Microbe”; Attorney Docket No. 11836-045-999), filed Jun. 1, 2010. Representative Ime2p amino acid sequences of Saccharomyces cerevisiae include Genbank accession number NP_{—}012429 and sequences identified as SEQ ID NOS: 4, 32, 60, 88, and 116 in U.S. patent application Ser. No. ______ (entitled “Methods for Generating a Genetically Modified Microbe”; Attorney Docket No. 11836-045-999), filed Jun. 1, 2010.

In some embodiments, the sporulation gene is NDT80. The sequence of the NDT80 gene of Saccharomyces cerevisiae has been previously described. See, e.g., Goffeau et al., Science 274 (5287):546-547 (1996). Representative NDT80 nucleotide sequences of Saccharomyces cerevisiae include Genbank accession number NC_{—}001140 and sequences identified as SEQ ID NOS: 5, 33, 61, 89, and 117 in U.S. patent application Ser. No. ______ (entitled “Methods for Generating a Genetically Modified Microbe”; Attorney Docket No. 11836-045-999), filed Jun. 1, 2010. Representative Ndt80p amino acid sequences of Saccharomyces cerevisiae include Genbank accession number NP_{—}011992 and sequences identified as SEQ ID NOS: 6, 34, 62, 90, and 118 in U.S. patent application Ser. No. ______ (entitled “Methods for Generating a Genetically Modified Microbe”; Attorney Docket No. 11836-045-999), filed Jun. 1, 2010.

In some embodiments, the sporulation gene is SPO11. The sequence of the SPO11 gene of Saccharomyces cerevisiae has been previously described. See, e.g., Atcheson et al., Proc. Natl. Acad. Sci. U.S.A. 84 (22), 8035-8039 (1987). Representative SPO11 nucleotide sequences of Saccharomyces cerevisiae include Genbank accession number J02987 and sequences identified as SEQ ID NOS: 7, 35, 63, 91, and 119 in U.S. patent application Ser. No. ______ (entitled “Methods for Generating a Genetically Modified Microbe”; Attorney Docket No. 11836-045-999), filed Jun. 1, 2010. Representative Spo11p amino acid sequences of Saccharomyces cerevisiae include Genbank accession number AAA65532 and sequences identified as SEQ ID NOS: 8, 36, 64, 92, and 120 in U.S. patent application Ser. No. ______ (entitled “Methods for Generating a Genetically Modified Microbe”; Attorney Docket No. 11836-045-999), filed Jun. 1, 2010.

In some embodiments, the sporulation gene is SPO20. The sequence of the SPO20 gene of Saccharomyces cerevisiae has been previously described. See, e.g., Bowman et al., Nature 387 (6632 Suppl), 90-93 (1997). Representative SPO20 nucleotide sequences of Saccharomyces cerevisiae include Genbank accession number AF078740 and sequences identified as SEQ ID NOS: 9, 37, 65, 93, and 121 in U.S. patent application Ser. No. ______ (entitled “Methods for Generating a Genetically Modified Microbe”; Attorney Docket No. 11836-045-999), filed Jun. 1, 2010. Representative Spo20p amino acid sequences of Saccharomyces cerevisiae include Genbank accession number NP_{—}013730 and sequences identified as SEQ ID NOS: 10, 38, 66, 94, and 122 in U.S. patent application Ser. No. ______ (entitled “Methods for Generating a Genetically Modified Microbe”; Attorney Docket No. 11836-045-999), filed Jun. 1, 2010.

In some embodiments, the sporulation gene is AMA1. The sequence of the AMA1 gene of Saccharomyces cerevisiae has been previously described. See, e.g., Tettelin et al., Nature 387 (6632 Suppl):81-84 (1997). Representative AMA1 nucleotide sequences of Saccharomyces cerevisiae include Genbank accession number NC_{—}001139.8 and sequences identified as SEQ ID NOS: 11, 39, 67, 95, and 123 in U.S. patent application Ser. No. ______ (entitled “Methods for Generating a Genetically Modified Microbe”; Attorney Docket No. 11836-045-999), filed Jun. 1, 2010. Representative Ama1p amino acid sequences of Saccharomyces cerevisiae include Genbank accession number NP_{—}011741 and sequences identified as SEQ ID NOS: 12, 40, 68, 96, and 124 in U.S. patent application Ser. No. ______ (entitled “Methods for Generating a Genetically Modified Microbe”; Attorney Docket No. 11836-045-999), filed Jun. 1, 2010.

In some embodiments, the sporulation gene is HOP2. The sequence of the HOP2 gene of Saccharomyces cerevisiae has been previously described. See, e.g., Leu et al., Cell 94 (3):375-386 (1998). Representative HOP2 nucleotide sequences of Saccharomyces cerevisiae include Genbank accession number AF_{—}078740.1 and sequences identified as SEQ ID NOS: 13, 41, 69, 97, and 125 in U.S. patent application Ser. No. ______ (entitled “Methods for Generating a Genetically Modified Microbe”; Attorney Docket No. 11836-045-999), filed Jun. 1, 2010. Representative Hop2p amino acid sequences of Saccharomyces cerevisiae include Genbank accession number AAC31823 and sequences identified as SEQ ID NOS: 14, 42, 70, 98, and 126 in U.S. patent application Ser. No. ______ (entitled “Methods for Generating a Genetically Modified Microbe”; Attorney Docket No. 11836-045-999), filed Jun. 1, 2010.

In some embodiments, the sporulation gene is SPO21. The sequence of the SPO21 gene of Saccharomyces cerevisiae has been previously described. See, e.g., Dujon et al., Nature 387 (6632 Suppl):98-102 (1997). Representative SPO21 nucleotide sequences of Saccharomyces cerevisiae include Genbank accession number NC_{—}001147.5 and sequences identified as SEQ ID NOS: 15, 43, 71, 99, and 127 in U.S. patent application Ser. No. ______ (entitled “Methods for Generating a Genetically Modified Microbe”; Attorney Docket No. 11836-045-999), filed Jun. 1, 2010. Representative Spo21p amino acid sequences of Saccharomyces cerevisiae include Genbank accession number NP_{—}014550 and sequences identified as SEQ ID NOS: 16, 44, 72, 100, and 128 in U.S. patent application Ser. No. ______ (entitled “Methods for Generating a Genetically Modified Microbe”; Attorney Docket No. 11836-045-999), filed Jun. 1, 2010.

5.2.5 Methods for Genetically Modifying Microbes

Methods for genetically modifying microbes using expression vectors or chromosomal integration constructs are well known in the art. See, for example, Sherman, F., et al., Methods Yeast Genetics, Cold Spring Harbor Laboratory, N.Y. (1978); Guthrie, C., et al. (Eds.) Guide To Yeast Genetics and Molecular Biology Vol. 194, Academic Press, San Diego (1991); Sambrook et al., 2001, Molecular Cloning—A Laboratory Manual, 3^{rd} edition, Cold Spring Harbor Laboratory, Cold Spring Harbor, N.Y.; and Ausubel et al., eds., Current Edition, Current Protocols in Molecular Biology, Greene Publishing Associates and Wiley Interscience, NY.; the disclosures of which are incorporated herein by reference.

5.2.5.1 Expression Vectors

In some embodiments, the methods of the present invention require the use of expression vectors to express in the microbe a particular protein. Generally, expression vectors are recombinant polynucleotide molecules comprising replication signals and expression control sequences, e.g., promoters and terminators, operatively linked to a nucleotide sequence encoding a polypeptide. Expression vectors useful for expressing polypeptide-encoding nucleotide sequences include viral vectors (e.g., retroviruses, adenoviruses and adenoassociated viruses), plasmid vectors, and cosmids. Illustrative examples of expression vectors suitable for use in yeast cells include, but are not limited to CEN/ARS and 2μ plasmids. Illustrative examples of promoters suitable for use in yeast cells include, but are not limited to the promoter of the TEF1 gene of K. lactis, the promoter of the PGK1 gene of Saccharomyces cerevisiae, the promoter of the TDH3 gene of Saccharomyces cerevisiae, repressible promoters, e.g., the promoter of the CTR3 gene of Saccharomyces cerevisiae, and inducible promoters, e.g., galactose inducible promoters of Saccharomyces cerevisiae (e.g., promoters of the GAL1, GAL7, and GAL10 genes).

5.2.5.2 Chromosomal Integration Constructs

In some embodiments, the methods of the present invention require the use of one or more chromosomal integration constructs for the stable introduction of a heterologous nucleotide sequence into a specific location in a chromosome or for the functional disruption of one or more target sporulation genes and/or one or more target pheromone response genes in a genetically modified microbial cell. In some embodiments, disruption of the target gene prevents the expression of a functional protein. In some embodiments, disruption of the target gene results in expression of a non-functional protein from the disrupted gene.

In some embodiments, the chromosomal integration construct is a linear DNA molecule. In other embodiments, the chromosomal integration construct is a circular DNA molecule. In some embodiments, the circular or linear disruption construct comprises a pair of homologous sequences, i.e., nucleotide sequences that are homologous to nucleotide sequences at the locus in the chromosome to which the integrating sequence is targeted (target locus), e.g., a target gene, separated by an integrating sequence. In some embodiments, the circular chromosomal integration construct comprises a single homologous sequence. Such circular chromosomal integration constructs, upon integration at the target locus, would become linearized, with a portion of the homologous sequence positioned at each end and the remaining segments of the chromosomal integration construct inserting into the target locus without replacing any of the target locus nucleotide sequence. In particular embodiments, the single homologous sequence of a circular chromosomal integration construct is homologous to a sequence located within the coding sequence of a target gene.

Parameters of chromosomal integration constructs that may be varied in the practice of the present invention include, but are not limited to, the lengths of the homologous sequences; the nucleotide sequence of the homologous sequences; the length of the integrating sequence; the nucleotide sequence of the integrating sequence; and the nucleotide sequence of the target locus.

In some embodiments, an effective range for the length of each homologous sequence is 50 to 5,000 base pairs. In particular embodiments, the length of each homologous sequence is about 500 base pairs. For a discussion of the length of homology required for gene targeting, see Hasty et al., Mol Cell Biol 11:5586-91 (1991).

In some embodiments, the homologous sequences comprise coding sequences of a target gene. In other embodiments, the homologous sequences comprise upstream or downstream sequences of a target gene. In some embodiments, one homologous sequence comprises a nucleotide sequence that is homologous to a nucleotide sequence located within or 5′ of the coding sequence of a target gene, and the other homologous sequence comprises a nucleotide sequence that is homologous to a nucleotide sequence located 3′ of the coding sequence of a target gene. In some embodiments, one homologous sequence comprises a nucleotide sequence that is homologous to a nucleotide sequence located 5′ of the coding sequence of a target gene, and the other homologous sequence comprises a nucleotide sequence that is homologous to a nucleotide sequence located within or 3′ of the coding sequence of a target gene. In some embodiments, both homologous sequences comprise nucleotide sequences that are homologous to nucleotide sequences located within the coding sequence of a target gene. In some embodiments, one homologous sequence comprises a nucleotide sequence that is homologous to a nucleotide sequence located 5′ of the coding sequence of a target gene, and the other homologous sequence comprises a nucleotide sequence that is homologous to a nucleotide sequence located within the coding sequence of a target gene, and the integrating sequence comprises a nucleotide sequence encoding a promoter that can be induced or repressed by addition of an inducer or repressor, respectively, to the culture medium in which the microbial cell is cultivated, such that upon integration of the integrating sequence at the target locus the promoter of the target gene is replaced with the inducible or repressible promoter, rendering production of the target gene product dependent on the presence of the inducing or repressing agent in the culture medium.

In some embodiments, the length for the integrating sequence is from 1 to 10,000 base pairs. In some embodiments, the length for the integrating sequence is from 1 to 8,000 base pairs. In some embodiments, the length for the integrating sequence is from 1 to 6,000 base pairs. In some embodiments, the length for the integrating sequence is from 1 to 4,000 base pairs. In some embodiments, the length for the integrating sequence is from 1 to 2,000 base pairs. In some embodiments, the length for the integrating sequence is a length approximately equivalent to the distance between the regions of the target locus that match the homologous sequences in the chromosomal integration construct.

In some embodiments, the integrating sequence comprises a nucleotide sequence encoding a selectable marker that enables selection of microbial cells comprising the integrating sequence. In some embodiments, the integrating sequence comprises a nucleotide sequence encoding one or more proteins of interest. In some embodiments, a termination codon is positioned in-frame with and downstream of the nucleotide sequence encoding the selectable marker and/or protein of interest to prevent translational read-through that might yield a fusion protein.

5.2.5.3 Selectable Markers

In some embodiments, the expression vector or chromosomal integration vector used to genetically modify a microbial cell of the invention comprises one or more selectable markers useful for the selection of transformed microbial cells.

In some embodiments, the selectable marker is an antibiotic resistance marker. Illustrative examples of antibiotic resistance markers include, but are not limited to the BLA, NAT1, PAT, AUR1-C, PDR4, SMR1, CAT, mouse dhfr, HPH, DSDA, KAN^{R}, and SH BLE gene products. The BLA gene product from E. coli confers resistance to beta-lactam antibiotics (e.g., narrow-spectrum cephalosporins, cephamycins, and carbapenems (ertapenem), cefamandole, and cefoperazone) and to all the anti-gram-negative-bacterium penicillins except temocillin; the NAT1 gene product from S. noursei confers resistance to nourseothricin; the PAT gene product from S. viridochromogenes Tu94 confers resistance to bialophos; the AUR1-C gene product from Saccharomyces cerevisiae confers resistance to Auerobasidin A (AbA); the PDR4 gene product confers resistance to cerulenin; the SMR1 gene product confers resistance to sulfometuron methyl; the CAT gene product from Tn9 transposon confers resistance to chloramphenicol; the mouse dhfr gene product confers resistance to methotrexate; the HPH gene product of Klebsiella pneumonia confers resistance to Hygromycin B; the DSDA gene product of E. coli allows cells to grow on plates with D-serine as the sole nitrogen source; the KAN^{R} gene of the Tn903 transposon confers resistance to G418; and the SH BLE gene product from Streptoalloteichus hindustanus confers resistance to Zeocin (bleomycin). In some embodiments, thes antibiotic resistance marker is deleted after the genetically modified microbial cell of the invention is isolated.

In some embodiments, the selectable marker rescues an auxotrophy (e.g., a nutritional auxotrophy) in the genetically modified microbial cell. In such embodiments, a parent microbial cell comprises a functional disruption in one or more gene products that function in an amino acid or nucleotide biosynthetic pathway, such as, for example, the HIS3, LEU2, LYS1, LYS2, MET15, TRP1, ADE2, and URA3 gene products in yeast, which renders the parent microbial cell incapable of growing in media without supplementation with one or more nutrients (auxotrophic phenotype). The auxotrophic phenotype can then be rescued by transforming the parent microbial cell with an expression vector or chromosomal integration encoding a functional copy of the disrupted gene product, and the genetically modified microbial cell generated can be selected for based on the loss of the auxotrophic phenotype of the parent microbial cell. Utilization of the URA3, TRP1, and LYS2 genes as selectable markers has a marked advantage because both positive and negative selections are possible. Positive selection is carried out by auxotrophic complementation of the URA3, TRP1, and LYS2 mutations, whereas negative selection is based on specific inhibitors, i.e., 5-fluoro-orotic acid (FOA), 5-fluoroanthranilic acid, and a-aminoadipic acid (aAA), respectively, that prevent growth of the prototrophic strains but allows growth of the URA3, TRP1, and LYS2 mutants, respectively.

In other embodiments, the selectable marker rescues other non-lethal deficiencies or phenotypes that can be identified by a known selection method.

5.2.5.4 Microbial Cell Transformations

Expression vectors and chromosomal integration constructs can be introduced into microbial cells by any method known to one of skill in the art without limitation. See, for example, Hinnen et al., Proc. Natl. Acad. Sci. USA 75:1292-3 (1978); Cregg et al., Mol. Cell. Biol. 5:3376-3385 (1985); U.S. Pat. No. 5,272,065; Goeddel et al., eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY. Exemplary techniques include, but are not limited to, spheroplasting, electroporation, PEG 1000 mediated transformation, and lithium acetate or lithium chloride mediated transformation.

5.2.6 Methods for Culturing Genetically Modified Microbes

The present invention provides methods for producing an isoprenoid compound. The methods generally involve culturing genetically modified microbial cells of the invention under suitable conditions in a suitable medium comprising a carbon source.

Suitable conditions and suitable media for culturing microbial cells are well known in the art. In some embodiments, the suitable medium is supplemented with one or more additional agents, such as, for example, an inducer (e.g., when one or more nucleotide sequences encoding a gene product is under the control of an inducible promoter), a repressor (e.g., when one or more nucleotide sequences encoding a gene product are under the control of a repressible promoter), or a selection agent (e.g., an antibiotic to select for microbial cells comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limited examples of suitable non-fermentable carbon sources include acetate and glycerol.

5.2.7 Methods for Extracting Isoprenoid Compounds from Fermentation

The isoprenoid compound produced by the genetically modified microbial cells may be isolated from the fermentation using any suitable separation and purification methods known in the art.

In some embodiments, an organic phase comprising the isoprenoid compound is separated from the fermentation by centrifugation. In other embodiments, an organic phase comprising the isoprenoid compound separates from the fermentation spontaneously. In yet other embodiments, an organic phase comprising the isoprenoid compound is separated from the fermentation by adding a deemulsifier and/or a nucleating agent into the fermentation reaction. Illustrative examples of deemulsifiers include flocculants and coagulants. Illustrative examples of nucleating agents include droplets of the isoprenoid compound itself and organic solvents such as dodecane, isopropyl myristrate, and methyl oleate.

In some embodiments, the isoprenoid compound is separated from other products that may be present in the organic phase. In some embodiments, separation is achieved using adsorption, distillation, gas-liquid extraction (stripping), liquid-liquid extraction (solvent extraction), ultrafiltration, and standard chromatographic techniques.

In some embodiments, the isoprenoid compound is pure, e.g., at least about 40% pure, at least about 50% pure, at least about 60% pure, at least about 70% pure, at least about 80% pure, at least about 90% pure, at least about 95% pure, at least about 98% pure, or more than 98% pure, where “pure” in the context of an isoprenoid compound refers to an isoprenoid compound that is free from other isoprenoid compounds, contaminants, etc.

5.2.8 Generation of Diploid Yeast Cells

Certain methods provided herein comprise a step of inducing mating among haploid cells that comprise a functional disruption in one or more sporulation genes and/or a functional disruption in one or more pheromone response genes. The diploid cells formed as a result of said mating are stable diploid cells constrained to the diploid phase due to the functional disruption of the one or more sporulation genes of the cell.

To form a diploid cell from haploid cells that lack mating capability, the mating-impaired haploid cells are transformed with a “mating complement plasmid,” i.e., a recombinant plasmid comprising a heterologous gene that can complement the mating deficiency caused by the functional disruption in the one or more pheromone response genes. Transient expression of the heterologous pheromone response gene within the haploid cells temporarily restores mating function to the cells and enables haploid cells of opposite mating type to form a stable diploid cell. In particular, the stable diploid cells formed thereby are homozygous other than for their mating type allele, being generated from haploids of the same genetically modified population.

Thus, in some embodiments in which the haploid cell comprises a functional disruption of the STE5 gene, the haploid cell is transformed with a mating complement plasmid comprising a STE5 coding sequence. In some embodiments in which the haploid cell comprises a functional disruption of the STE4 gene, the haploid cell is transformed with a mating complement plasmid comprising a STE4 coding sequence. In some embodiments in which the haploid cell comprises a functional disruption of the STE18 gene, the haploid cell is transformed with a mating complement plasmid comprising a STE18 coding sequence. In embodiments in which the haploid cell comprises a functional disruption of the STE12 gene, the haploid cell is transformed with a mating complement plasmid comprising a STE12 coding sequence. In embodiments in which the a haploid cell comprises a functional disruption of the STE7 gene, the haploid cell is transformed with a mating complement plasmid encoding a STE7 coding sequence. In some embodiments in which the haploid cell comprises a functional disruption of the STE11 gene, the haploid cell is transformed with a mating complement plasmid comprising a STE11 coding sequence.

Plasmid-based systems generally require selective pressure on the plasmids to maintain the foreign DNA in the cell. Most plasmids in yeast are relatively unstable, as a yeast cell typically loses 10% of plasmids contained in the cell after each mitotic division. Thus, in some embodiments, selection of diploid cells that were formed by the mating of haploid cells comprising a plasmid encoding a mating complement gene but that do not themselves comprise the plasmid is achieved by allowing the diploid cells to undergo sufficient mitotic divisions such that the plasmid is effectively diluted from the population. Alternatively, diploid cells can be selected by selecting for the absence of the plasmid, e.g., by selecting against a counter-selectable marker (such as, for example, URA3) or by plating identical colonies on both selective media and non-selective media and then selecting a colony that does not grow on the selective media but does grow on the non-selective media.

In some embodiments, the methods provided herein comprise a step of transforming a haploid heterothallic (ho) yeast cell with a recombinant plasmid encoding a homothallism (HO) protein, wherein expression of the HO protein induces a mating-type switch of the haploid cell. The sequence of the HO gene of Saccharomyces cerevisiae has been previously described. See, e.g., Russell et al., Mol. Cell. Biol. 6 (12):4281-4294 (1986). Representative HO nucleotide sequences of Saccharomyces cerevisiae include Genbank accession number NC_{—}001136.

5.2.9 Detection of Microbial Cells of the Invention

Provided herein are methods for detecting in a biological sample the presence or absence of a genetically modified microbial cell comprising one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17. The methods employ a first compound or agent that is capable of interacting with a target molecule, followed by detection of said interaction, wherein the target molecule is either a nucleic acid comprising a nucleotide sequence encoding a polypeptide having an amino acid sequence that is at least 80% identical to an amino acid sequence selected from the group consisting of SEQ ID NOs: 1, 3, 5, 7, 9, 11, 13, 15, and 17, or a polypeptide having an amino acid sequence that is at least 80% identical to an amino acid sequence selected from the group consisting of SEQ ID NOs: 1, 3, 5, 7, 9, 11, 13, 15, and 17.

In some embodiments, the first compound or agent is a nucleic acid probe that can hybridize to a nucleic acid encoding a polypeptide having an amino acid sequence that is at least 80% identical to an amino acid sequence selected from the group consisting of SEQ ID NOs: 1, 3, 5, 7, 9, 11, 13, 15, and 17. The nucleic acid probe can comprise the entire nucleotide sequence encoding the polypeptide or a portion thereof (e.g., at least 10, 15, 30, 50, 100, 250, or 500 nucleotides in length) that is sufficient to specifically hybridize under stringent conditions to a nucleic acid encoding the polypeptide. In some embodiments, the nucleic acid probe is physically linked to a detectable substance. Illustrative examples of detectable substances include, but are not limited to fluorescent molecules, biotin, and radioactive isotopes.

In other embodiments, the first compound or agent is an antibody or an antibody fragment that can bind a polypeptide having an amino acid sequence that is at least 80% identical to an amino acid sequence selected from the group consisting of SEQ ID NOs: 1, 3, 5, 7, 9, 11, 13, 15, and 17. In some embodiments, the antibody or antibody fragment is polyclonal. In other such embodiments, the antibody or antibody fragment is monoclonal. In some embodiments, the antibody fragment is a Fab fragment. In some embodiments, the antibody or antibody fragment is physically linked to a detectable substance. Illustrative examples of detectable substances include, but are not limited to fluorescent molecules, biotin, and radioactive isotopes.

In some embodiments, detecting the interaction of the first compound or agent with the target molecule is achieved by detecting the detectable substance that is physically linked to the first compound or agent. In other embodiments, detecting the interaction of the first compound or agent with the target molecule is achieved by contacting the biological sample with a second compound or agent that is physically linked to a detectable substance, and detecting the detectable substance that is physically linked to the second compound or agent, wherein the second compound or agent is capable of interacting with the first compound or agent.

Well known methods for detecting nucleic acids and polypeptides in a biological sample include, but are not limited to nucleic acid hybridizations (e.g., Southern blot hybridization, Northern blot hybridization, in situ hybridization, fluorescence in situ hybridization (FISH)), antibody binding assays (e.g., Western blot hybridization, enzyme linked immunosorbent assays (ELISAs), immunoprecipitations, immunofluorescence), and PCR-based methods.

By way of example and not limitation, a nucleic acid hybridization under stringent conditions may proceed as follows: Prehybridization of filters containing DNA may be carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters may be hybridized for 48 hours at 65° C. in prehybridization mixture containing 100 μg/ml denatured salmon sperm DNA and 5-20×10⁶ cpm of ³²P-labeled nucleic acid probe. Washing of filters may be done at 37° C. for 1 hour in a solution containing 2×SSC, 0.01% PVP, 0.01% Ficoll, and 0.01% BSA. This can be followed by a wash in 0.1×SSC at 50° C. for 45 minutes before autoradiography. Other conditions for stringent hybridization that may be used are well known in the art.

### 6. EXAMPLES

### 6.1 Example 1

### Generation of Genetically Modified Haploid Cells

This example describes an exemplary method for generating genetically modified haploid S. cerevisiae cells.

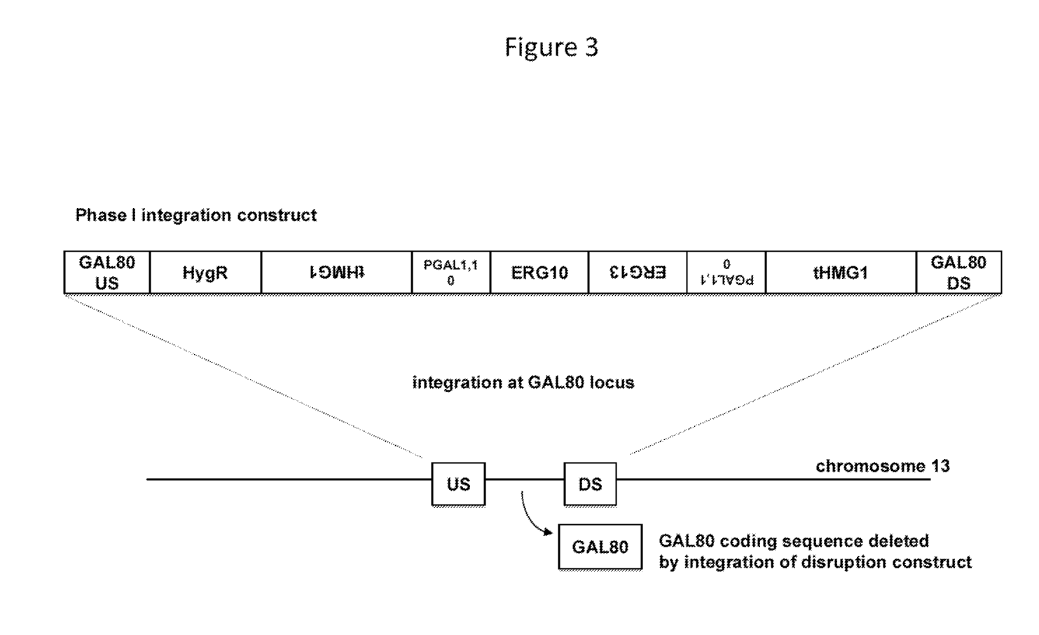

The Phase I integration construct (FIG. 3; SEQ ID NO: 141) comprises as an integrating sequence nucleotide sequences that encode a selectable marker (hygA, which confers resistance to hygromycin B); two enzymes of the S. cerevisiae MEV pathway (the truncated HMG1 coding sequence, which encodes a truncated HMG-CoA reductase, and the ERG13 coding sequence, which encodes HMG-CoA synthase), and another enzyme of S. cerevisiae (the ERG10 coding sequence, which encodes acetoacetyl-CoA thiolase), under control of galactose-inducible promoters (promoters of the S. cerevisiae genes GAL1 and GAL10); flanked by homologous sequences consisting of upstream and downstream nucleotide sequences of the S. cerevisiae GAL80 locus. Upon introduction into a S. cerevisiae host cell, the Phase I integration construct can integrate by homologous recombination into the GAL80 locus of the S. cerevisiae host cell genome, and functionally disrupt the GAL80 locus by replacing the GAL80 coding sequence with its integrating sequence. The Phase I integration construct was cloned into the TOPO Zero Blunt II cloning vector (Invitrogen, Carlsbad, Calif.), yielding plasmid TOPO-Phase I integration construct. The construct was propagated in TOP10 cells grown on LB agar containing 50 μg/ml kanamycin.

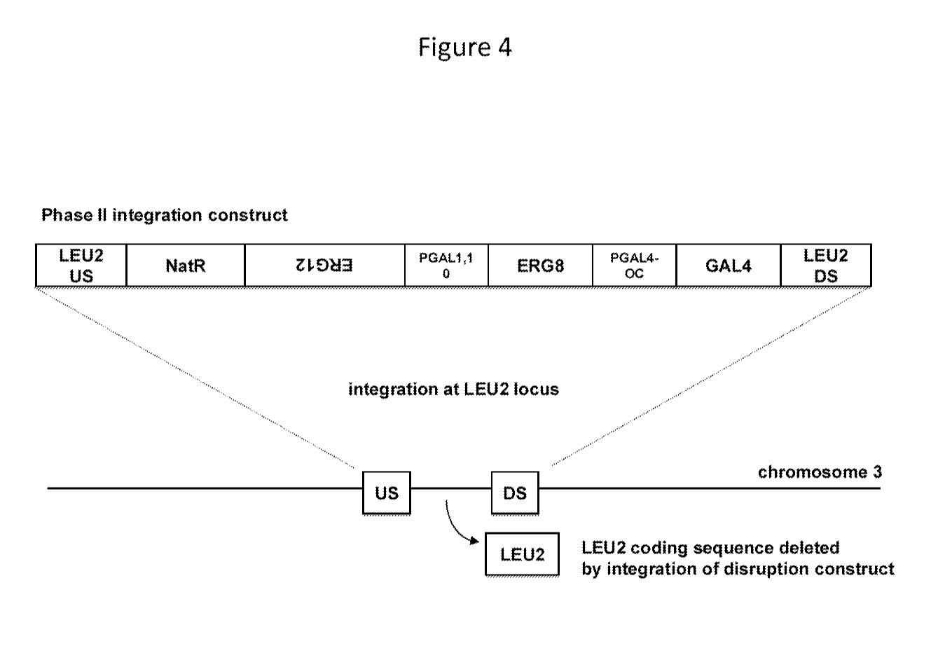

The Phase II integration construct (FIG. 4; SEQ ID NO: 142) comprises as an integrating sequence nucleotide sequences encoding a selectable marker (natA, which confers resistance to nourseothricin) and several enzymes of the S. cerevisiae MEV pathway (the ERG12 coding sequence, which encodes mevalonate kinase, and the ERG8 coding sequence, which encodes phosphomevalonate kinase), under control of galactose-inducible promoters (promoters of the S. cerevisiae genes GAL1 and GAL10); as well as the coding sequence of the S. cerevisiae GAL4 gene under control of the GAL4oc promoter (GAL4 promoter comprising a mutation that removes the MIG1 binding site thus making the promoter less sensitive to the repression by glucose); flanked by homologous sequences consisting of upstream and downstream nucleotide sequences of the S. cerevisiae LEU2 locus. Upon introduction into a S. cerevisiae host cell, the Phase II integration construct can integrate by homologous recombination into the LEU2 locus of the S. cerevisiae host cell genome, and functionally disrupt the LEU2 locus by replacing the LEU2 coding sequence with its integrating sequence. The Phase II integration construct was cloned into the TOPO Zero Blunt II cloning vector, yielding plasmid TOPO-Phase II integration construct. The construct was propagated in TOP10 cells (Invitrogen, Carlsbad, Calif.) grown on LB agar containing 50 μg/ml kanamycin.

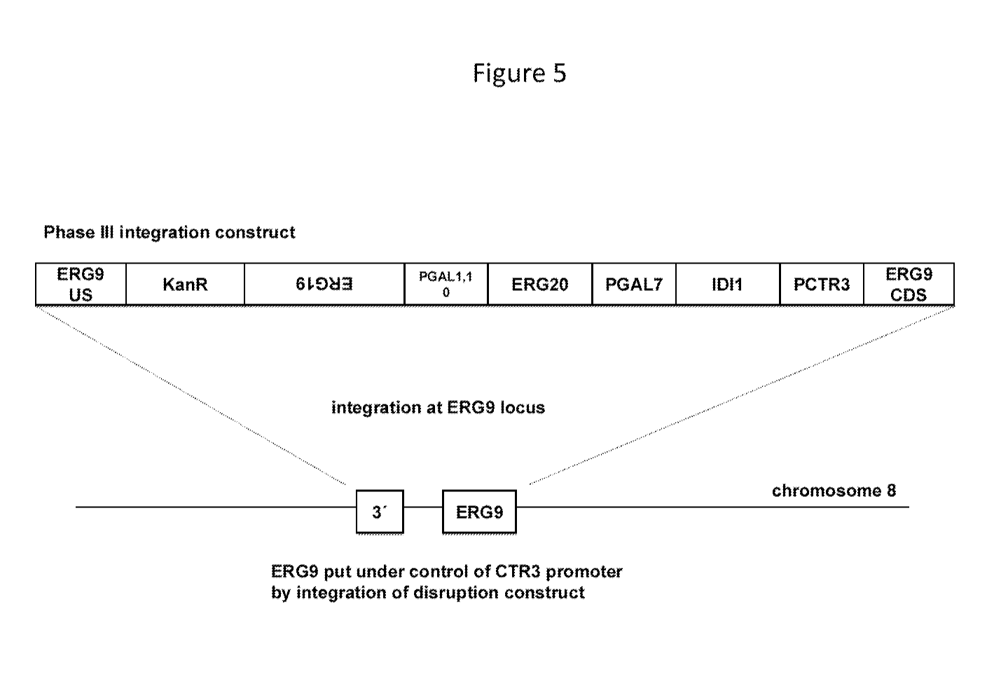

The Phase III integration construct (FIG. 5; SEQ ID NO: 143) comprises as an integrating sequence nucleotide sequences encoding a selectable marker (kanA, which confers resistance to G418); an enzyme of the S. cerevisiae MEV pathway (the ERG19 coding sequence, which encodes diphosphomevalonate decarboxylase), and two enzymes of S. cerevisiae involved in converting the product of the MEV pathway, IPP, into FPP (the ERG20 coding sequence, which encodes farnesyl pyrophosphate synthase, and the IDI1 coding sequence, which encodes isopentenyl pyrophosphate decarboxylase), under control of galactose-inducible promoters (promoters of the S. cerevisiae genes GAL1, GAL10, and GAL7); as well as the promoter of the S. cerevisiae CTR3 gene; flanked by upstream and coding nucleotide sequences of the S. cerevisiae ERG9 locus. Upon introduction into a S. cerevisiae host cell, the Phase II integration construct can integrate by homologous recombination upstream of the ERG9 locus of the S. cerevisiae host cell genome, replacing the native ERG9 promoter with the CTR3 promoter in such a way that the expression of ERG9 (squalene synthase) can be modulated by copper. The Phase III integration construct was cloned into the TOPO Zero Blunt II cloning vector, yielding plasmid TOPO-Phase III integration construct. The construct was propagated in TOP10 cells grown on LB agar containing 50 μg/ml kanamycin.

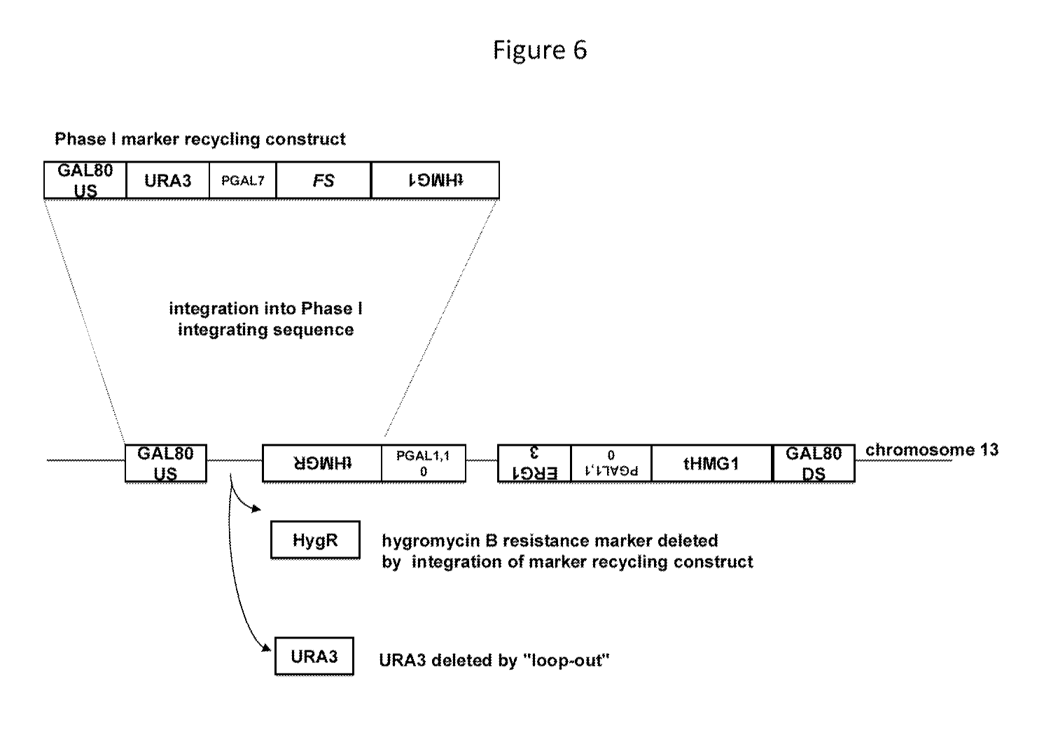

The Phase I marker recycling construct (FIG. 6; SEQ ID NO: 144) comprises nucleotide sequences encoding a selectable marker (URA3, which confers the ability to grow on media lacking uracil); and an enzyme of A. annua (the FS coding sequence, which encodes farnesene synthase), under regulatory control of the promoter of the S. cerevisiae GAL7 gene; flanked by upstream nucleotide sequences of the S. cerevisiae GAL80 locus and coding sequences of the S. cerevisiae HMG1 gene. Upon introduction into a S. cerevisiae host cell, the Phase I marker recycling construct can integrate by homologous recombination into the already integrated Phase I integrating sequence such that the selective marker hphA is replaced with URA3.

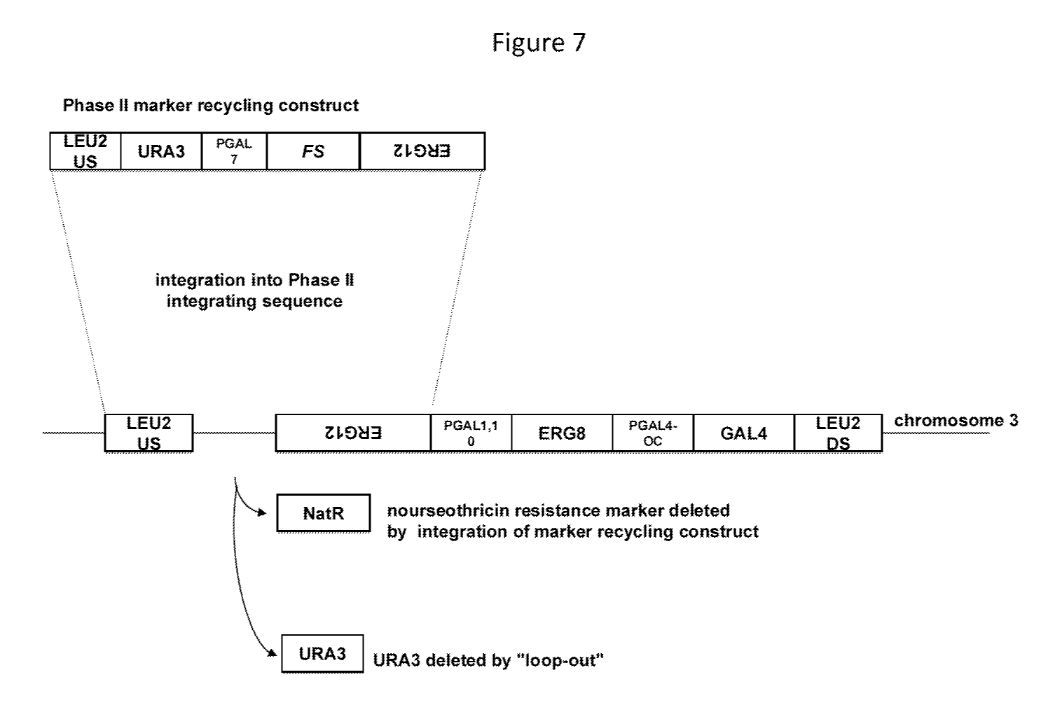

The Phase II marker recycling construct (FIG. 7; SEQ ID NO: 145) comprises nucleotide sequences encoding a selectable marker (URA3, which confers ability to grow on media lacking uracil) and an enzyme of A. annua (the FS coding sequence, which encodes farnesene synthase), under regulatory control of the promoter of the S. cerevisiae GAL7 gene; flanked by upstream nucleotide sequences of the S. cerevisiae LEU2 locus and coding sequences of the S. cerevisiae ERG12 gene. Upon introduction into a S. cerevisiae host cell, the Phase II marker recycling construct can integrate by homologous recombination into the already integrated Phase II integrating sequence such that the selective marker natA is replaced with URA3.

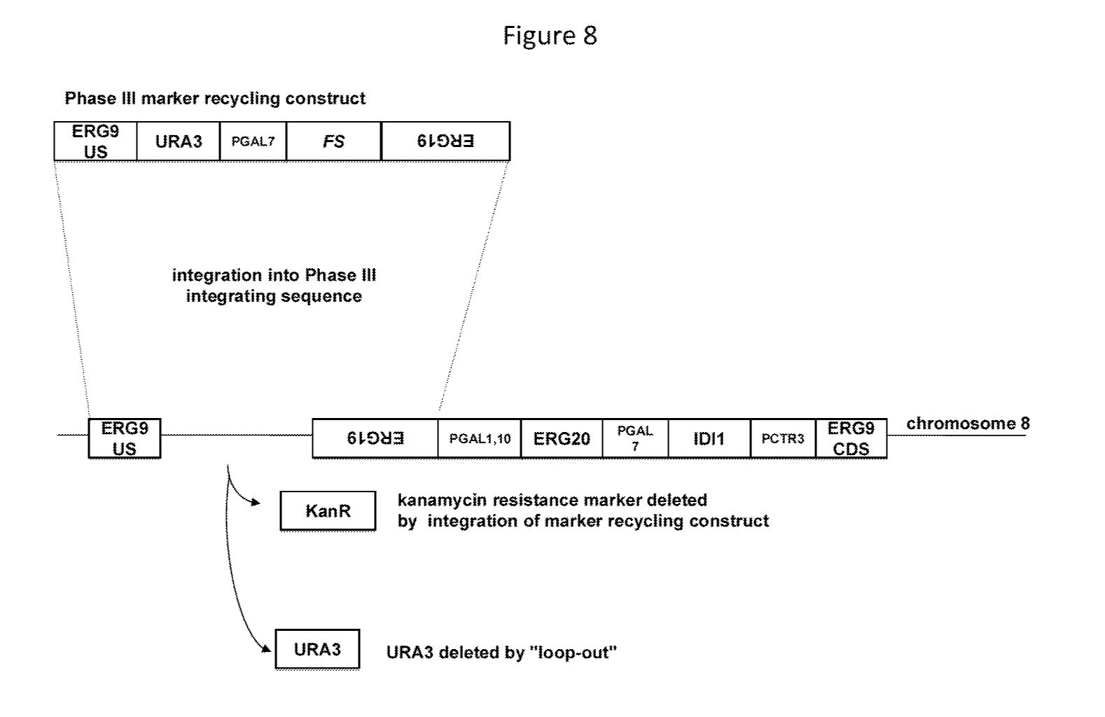

The Phase III marker recycling construct (FIG. 8; SEQ ID NO: 146) comprises nucleotide sequences encoding a selectable marker (URA3, which confers the ability to grow on media lacking uracil) and an enzyme of A. annua the FS coding sequence encodes farnesene synthase), under regulatory control of the promoter of the S. cerevisiae GAL7 gene; flanked by upstream nucleotide sequences of the S. cerevisiae ERG9 locus and coding sequences of the S. cerevisiae ERG19 gene. Upon introduction into a S. cerevisiae host cell, the Phase II marker recycling construct can integrate by homologous recombination into the already integrated Phase III integrating sequence such that the selective marker kanA is replaced with URA3.

Expression plasmid pAM404 (SEQ ID NO: 153) encodes a β-farnesene synthase. The nucleotide sequence insert was generated synthetically, using as a template the coding sequence of the β-farnesene synthase gene of Artemisia annua (GenBank accession number AY835398) codon-optimized for expression in Saccharomyces cerevisiae.

Starter host strain Y1198 was generated by resuspending active dry PE-2 yeast (isolated in 1994; gift from Santelisa Vale, Sertãozinho, Brazil) in 5 mL of YPD medium containing 100 ug/mL carbamicillin and 50 ug/mL kanamycin. The culture was incubated overnight at 30° C. on a rotary shaker at 200 rpm. An aliquot of 10 uL of the culture was then plated on a YPD plate and allowed to dry. The cells were serially streaked for single colonies, and incubated for 2 days at 30° C. Twelve single colonies were picked, patched out on a new YPD plate, and allowed to grow overnight at 30° C. The strain identities of the colonies were verified by analyzing their chromosomal sizes on a Bio-Rad CHEF DR II system (Bio-Rad, Hercules, Calif.) using the Bio-Rad CHEF Genomic DNA Plug Kit (Bio-Rad, Hercules, Calif.) according to the manufacturer's specifications. One colony was picked and stocked as strain Y1198.

Strains Y1661, Y1662, Y1663, and Y1664 were generated from strain Y1198 by rendering the strain haploid to permit genetic engineering. Strain Y1198 was grown overnight in 5 mL of YPD medium at 30° C. in a glass tube in a roller drum. The OD600 was measured, and the cells were diluted to an OD600 of 0.2 in 5 mL of YP medium containing 2% potassium acetate. The culture was grown overnight at 30° C. in a glass tube in a roller drum. The OD600 was measured again, and 4 OD600*mL of cells was collected by centrifugation at 5,000×g for 2 minutes. The cell pellet was washed once with sterile water, and then resuspended in 3 mL of 2% potassium acetate containing 0.02% raffinose. The cells were grown for 3 days at 30° C. in a glass tube in a roller drum. Sporulation was confirmed by microscopy. An aliquot of 33 μL of the culture was transferred to a 1.5 mL microfuge tube and was centrifuged at 14,000 rpm for 2 minutes. The cell pellet was resuspended in 50 μL of sterile water containing 2 μL of 10 mg/mL Zymolyase 100T (MP Biomedicals, Solon, Ohio), and the cells were incubated for 10 minutes in a 30° C. waterbath. The tube was transferred to ice, and 150 μL of ice cold water was added. An aliquot of 10 μL of this mixture was added to a 12 mL YPD plate, and tetrads were dissected on a Singer MSM 300 dissection microscope (Singer, Somerset, UK). The YPD plate was incubated at 30° C. for 3 days, after which spores were patched out onto a fresh YPD plate and grown overnight at 30° C. The mating types of each spore from 8 four-spore tetrads were analyzed by colony PCR. A single 4 spore tetrad with 2 MATa and 2 MATα spores was picked and stocked as strains Y1661 (MATa), Y1662 (MATa), Y1663 (MATα), and Y1664 (MATα).

For yeast cell transformations, 25 ml of Yeast Extract Peptone Dextrose (YPD) medium was inoculated with a single colony of a starting host strain. The culture was grown overnight at 30° C. on a rotary shaker at 200 rpm. The OD600 of the culture was measured, and the culture was then used to inoculate 50 ml of YPD medium to an OD600 of 0.15. The newly inoculated culture was grown at 30° C. on a rotary shaker at 200 rpm up to an OD600 of 0.7 to 0.9, at which point the cells were transformed with 1 μg of DNA. The cells were allowed to recover in YPD medium for 4 hours before they were plated on agar containing a selective agent to identify the host cell transformants.

Host strain Y1515 was generated by transforming strain Y1664 with plasmid TOPO-Phase I integration construct digested to completion using PmeI restriction endonuclease. Host cell transformants were selected on YPD medium containing 300 ug/mL hygromycin B, and positive transformants comprising the Phase. I integrating sequence integrated at the GAL80 locus were verified by the PCR amplification.

Host strain Y1762 was generated by transforming strain Y1515 with plasmid TOPO-Phase II integration construct digested to completion using PmeI restriction endonuclease. Host cell transformants were selected on YPD medium containing 100 ug/mL nourseothricin, and positive transformants comprising the Phase II integrating sequence integrated at the LEU2 locus were verified by the PCR amplification.

Host strain Y1770 was generated by transforming strain Y1762 in two steps with expression plasmid pAM404 and plasmid TOPO-Phase III integration construct digested to completion using PmeI restriction endonuclease. Host cell transformants with pAM404 were selected on Complete Synthetic Medium (CSM) lacking methionine and leucine. Host cell transformants with pAM404 and Phase III integration construct were selected on CSM lacking methionine and leucine and containing 200 ug/mL G418 (Geneticin®), and positive transformants comprising the Phase III integrating sequence integrated at the ERG9 locus were verified by the PCR amplification.

Host strain Y1793 was generated by transforming strain Y1770 with a URA3 knockout construct (SEQ ID NO: 154). The URA3 knockout construct comprises upstream and downstream sequences of the URA3 locus (generated from Saccharomyces cerevisiae strain CEN.PK2 genomic DNA). Host cell transformants were selected on YPD medium containing 5-FOA.

Host strain YAAA was generated by transforming strain Y1793 with the Phase I marker recycling construct. Host cell transformants were selected on CSM lacking methionine and uracil. The URA3 marker was excised by growing the cells overnight in YPD medium at 30° C. on a rotary shaker at 200 rpm, and then plating the cells onto agar containing 5-FOA. Marker excision was confirmed by colony PCR.

Host strain YBBB was generated by transforming strain YAAA with the Phase II marker recycling construct. Host cell transformants were selected on CSM lacking methionine and uracil. The URA3 marker was excised by growing the cells overnight in YPD medium at 30° C. on a rotary shaker at 200 rpm, and then plating the cells onto agar containing 5-FOA. Marker excision was confirmed by colony PCR.

Host strain Y1912 was generated by transforming strain YBBB with the Phase III marker recycling construct. Host cell transformants were selected on CSM lacking methionine and uracil. The URA3 marker was excised by growing the cells overnight in YPD medium at 30° C. on a rotary shaker at 200 rpm, and then plating the cells onto agar containing 5-FOA. Marker excision was confirmed by colony PCR.

### 6.2 Example 2

### Generation of Genetically Modified Sporulation and Endogenous Mating Impaired Haploid Cells

This example describes an exemplary method for disrupting a sporulation gene and a pheromone response gene in a genetically modified haploid S. cerevisiae cell to yield a genetically modified haploid S. cerevisiae cell that is sporulation and endogenous mating impaired.

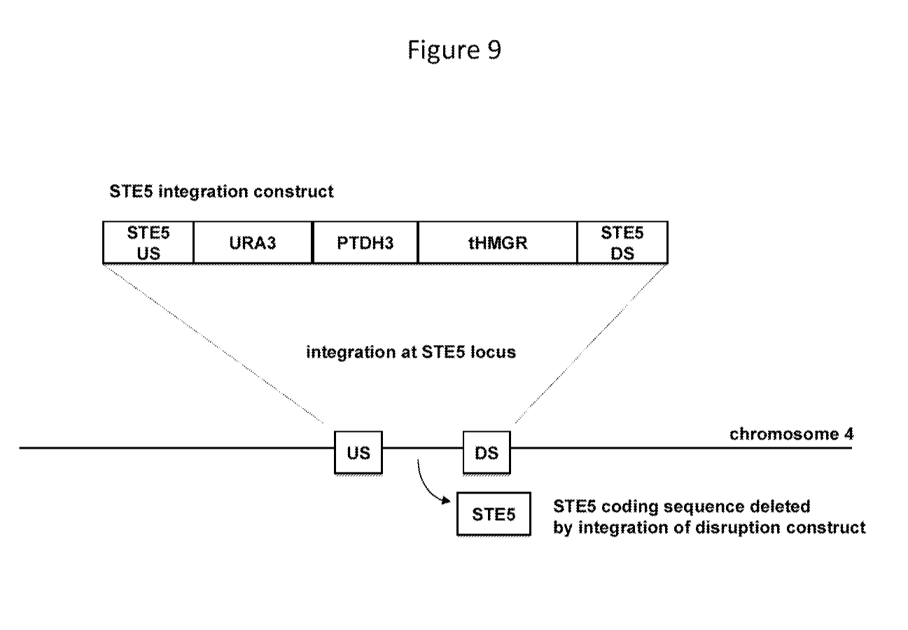

The STE5 integration construct (FIG. 9; SEQ ID NO: 147) comprises as an integrating sequence nucleotide sequences that encode a selectable marker (URA3, which confers ability to grow on media lacking uracil); and an enzyme of the S. cerevisiae MEV pathway (the truncated HMG1 coding sequence, which encodes a truncated HMG-CoA reductase), under regulatory control of the promoter of the S. cerevisiae TDH3 gene; flanked by homologous sequences consisting of upstream and downstream nucleotide sequences of the S. cerevisiae STE5 locus. Upon introduction into a S. cerevisiae host cell, the STE5 integration construct can integrate by homologous recombination into the STE5 locus of the S. cerevisiae host cell genome, functionally disrupting the STE5 locus by replacing the STE5 coding sequence with its integrating sequence.

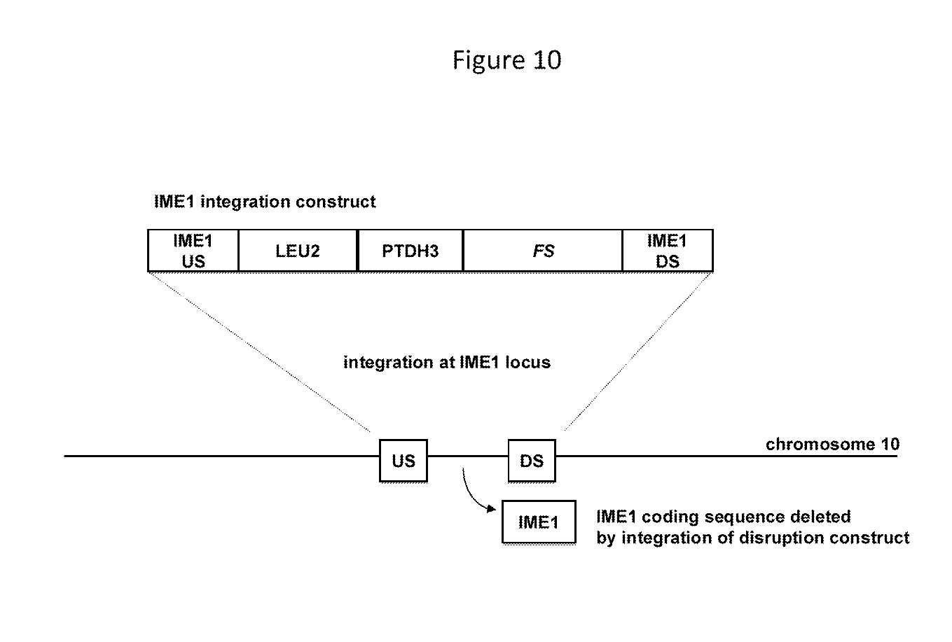

The IME1 integration construct (FIG. 10; SEQ ID NO: 148) comprises as an integrating sequence nucleotide sequences that encode a selectable marker (LEU2, which confers the ability to grow on media lacking leucine), and an enzyme of the A. annua (the FS coding sequence, which encodes a farnesene synthase), under regulatory control of the promoter of the S. cerevisiae TDH3 gene; flanked by homologous sequences consisting of upstream and downstream nucleotide sequences of the S. cerevisiae IME5 locus. Upon introduction into a S. cerevisiae host cell, the IME1 integration construct can integrate by homologous recombination into the IME1 locus of the S. cerevisiae host cell genome, functionally disrupting the IME1 locus by replacing the IME1 coding sequence with its integrating sequence.

Host strain Y1913 was generated by transforming strain Y1912 (see Example 1) with the STE5 integration construct. Host cell transformants were selected on CSM lacking methionine and uracil, and positive transformants were verified by PCR amplification.

Host strain Y1915 was generated from strain Y1913 by curing the strain from pAM404 and transforming the resulting strain with the IME1 integration construct. Strain Y1913 was propagated in non-selective YPD medium for 3 days at 30° C. on a rotary shaker at 200 rpm. Approximately 100 cells were plated onto YPD solid medium and allowed to grow for 3 days at 30° C. before they were replica-plated on CSM plates lacking methionine and leucine where they were grown for another 3 days at 30° C. Cured cells were identified by their ability to grow on minimal medium containing leucine and their inability to grow on medium lacking leucine. A single such colony was picked and transformed with the IME1 integration construct. Host cell transformants were selected on CSM lacking methionine and leucine.

### 6.3 Example 3

### Generation of Genetically Modified Sporulation and Endogenous Mating Impaired Diploid Cells

This example describes an exemplary method for rendering diploid a genetically modified haploid S. cerevisisea cell that is sporulation and endogenous mating impaired.

Diploid host strain Y1979 was generated by self-mating of strain Y1915. To generate cells of opposite mating types and to transiently render strain Y1915 capable of mating, the strain was co-transformed with plasmid pAM1124 (SEQ ID NO: 149), which encodes the HO protein and the nourseothricin resistance marker; and plasmid pAM1758 (SEQ ID NO: 150), which encodes STE5 and the G418 resistance marker. Host cell transformants were selected on CSM containing G418 and nourseothricin. Positive transformants were replated for single colonies on a non-selective medium, and G418 sensitive, nourseothricin sensitive diploids were identified through screening using colony PCR.

### 6.4 Example 4

### Confirmation of Sporulation and Endogenous Mating Impairment

This example describes exemplary methods with which to confirm the sporulation and endogenous mating impairment of genetically modified S. cerevisiae cells.

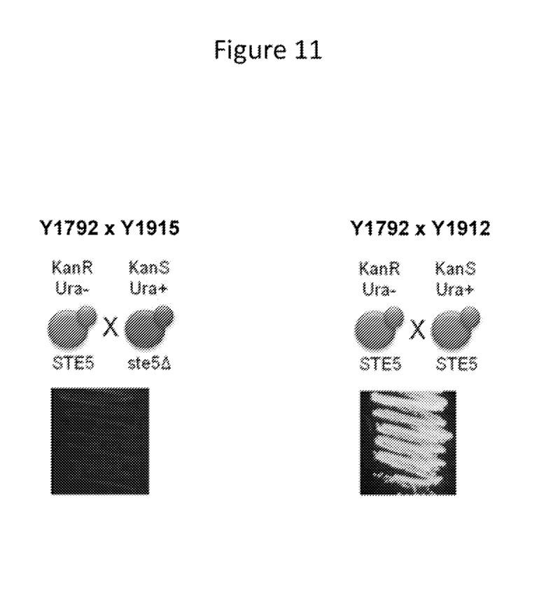

To confirm the inability of strain Y1915 to mate, haploid Y1915 cells (MATα Kan^{s} URA3 ̂ste5) or haploid Y1912 cells (MATα Kan^{S} URA3 STE5) were combined on YEPD solid medium with haploid Y1792 cells (MATa Kan^{R} ura3 STE5). The mating cultures were incubated for 16 hours at 30° C. Identical aliquots of each mating culture were then plated on CSM solid medium lacking uracil and containing G418, and the cultures were incubated for one week at 30° C. As shown in FIG. 11, colony growth was observed only on plates containing an aliquot of the Y1792×Y1912 mating culture but not on plates containing an aliquot of the Y1792×Y1915 mating culture.

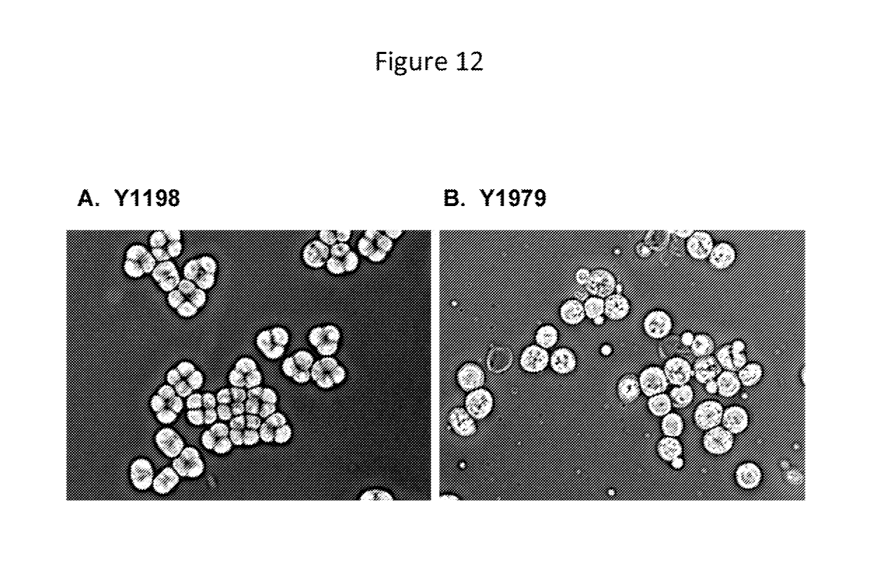

To confirm the inability of strain Y1979 to sporulate, strain Y1979 cells and strain Y1198 cells were cultivated for 7 days in sporulation induction medium (medium lacking a non-fermentative carbon source, e.g., potassium acetate, which induces native S. cerevisiae cells to abandon the cellular mitotic cycle and go into meiosis and sporulate). The cultures were then divided and treated for 15 minutes with water or diethyl ether. The suspensions were homogenized by inversion, re-suspended in sterile water, diluted, plated on YEPD solid medium, and grown for 3 days. As shown in FIG. 12, 95% of strain Y1198 cells formed tetrad spores under these conditions whereas strain Y1979 cells did not.

### 6.5 Example 5

### Confirmation of Inability of Sporulation and Endogenous Mating Impaired Cells to Disseminate in Nature

This example describes exemplary methods with which to confirm the inability of sporulation of endogenous mating impaired genetically modified diploid S. cerevisiae cells to disseminate in nature.

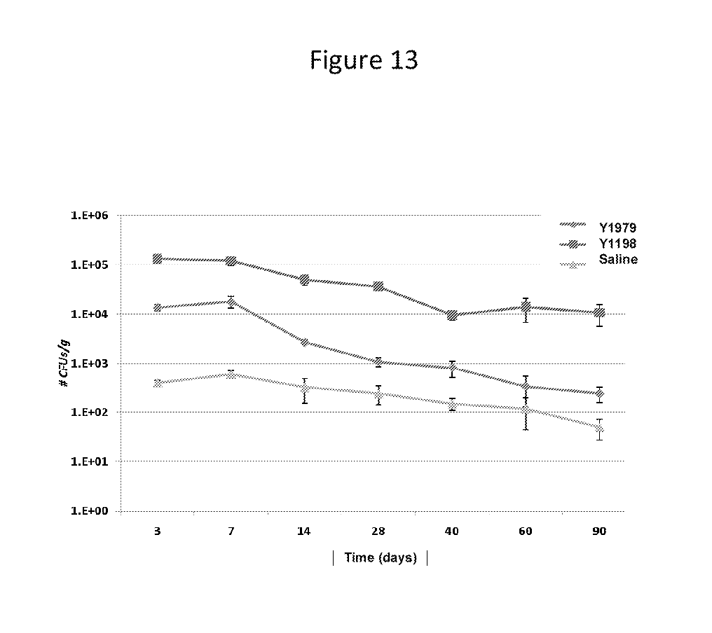

The survival of Y1979 and its non-transgenic isoline, Y1198 (PE-2), in soil was assessed. To this end, 45 L flasks were filled with approximately 25% vermiculite and 75% soil from the cane field (total of 40 L) and planted with 1 Saccharum spp, cultivar RB 86-7515 sugar cane plant (approximately 6 months old). Each pot was fertilized with a dry Nitrogen/Phosphorous/Potassium mix of 5-25-30, and the plants were grown for 14 days in a containment greenhouse. To each pot was then added 600 mL of cell suspensions of strain Y1979 or strain Y1198. The application of yeast cells is equivalent to attaining a concentration of 10⁷ cells/g in the first surface 5 cm of the soil. Five samples of 1.5×5 cm soil cores were collected at the following time points: t=0 (pre-exposure), 0 (post exposure), 3, 7, 14, 28, 40, 60, and 90 days (total volume of soil sampled was 44 mL, and total weight of soil sampled was approximately 50 g). From the composite samples, 10 grams were separated and resuspended in 100 mL of distilled water. To quantify yeast survival, 100 μL of the aqueous extractions were plated directly onto YPED medium (25 mL/plate), pH 5.5 adjusted with sulfuric acid 6N with addition of 0.05 g/L bengal rose (Sigma #R3877) and containing 0.2 g/L ampicillin (Sigma A0166). Samples were plated in duplicate, in dilution series from 1-10⁷, or the number of dilutions to be plated was based on the counts of survival obtained in the previous samplings for each treatment. Immediately after the plating the liquid was spread with a Drigalski spatula. The plates were left open to the flow for up to 30 minutes for total evaporation of the liquid and were then closed, inverted, and incubated for 48 hours at 30° C. The colony number per plate was read using a colony counter (CP600 Plus, Phoenix), in countable dilutions, and the result was expressed in CFU/plate. Counts were considered only if the total number of colonies was between 30-300 colonies. As shown in FIG. 13 (each data point is an average of five repetitions), Y1979 cells were clearly less viable in the soil than the genetically unmodified and sporulation and mating proficient parent cells of strain Y1198.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed:

1. A genetically modified yeast cell comprising:
(a) one or more heterologous nucleotide sequences encoding one or more enzymes of the mevalonate (MEV) pathway; and
(b) one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17.

2. The genetically modified yeast cell of claim 1, comprising a heterologous nucleotide sequence that encodes an enzyme that can convert HMG-CoA into mevalonate.

3. The genetically modified yeast cell of claim 1, comprising a heterologous nucleotide sequence that encodes an enzyme that can convert mevalonate into mevalonate 5-phosphate.

4. The genetically modified yeast cell of claim 1, wherein the one or more heterologous nucleotide sequences encodes more than one enzyme of the mevalonate pathway.

5. The genetically modified yeast cell of claim 1, comprising one or more nucleotide sequences encoding more than one polypeptide having an amino acid sequence that is at least 80% identical to an amino acid sequence selected from the group consisting of SEQ ID NOs: 1, 3, 5, 7, 9, 11, 13, 15, and 17.

6. The genetically modified yeast cell of claim 1, wherein the one or more nucleotide sequences are at least 85% identical to a nucleotide sequence selected from the group consisting of SEQ ID NOs: 2, 4, 6, 8, 10, 12, 14, 16, and 18.

7. The genetically modified yeast cell of claim 1, further comprising a heterologous nucleotide sequence encoding an enzyme that can convert isopentenyl pyrophosphate (IPP) into dimethylallyl pyrophosphate (DMAPP).

8. The genetically modified yeast cell of claim 1, further comprising a heterologous nucleotide sequence encoding an enzyme that can condense IPP and/or DMAPP molecules to form a polyprenyl compound.

9. The genetically modified yeast cell of claim 1, further comprising a heterologous nucleotide sequence encoding an enzyme that can modify IPP or a polyprenyl to form an isoprenoid compound.

10. The genetically modified yeast cell of claim 9, wherein the enzyme is selected from the group consisting of carene synthase, geraniol synthase, linalool synthase, limonene synthase, myrcene synthase, ocimene synthase, α-pinene synthase, β-pinene synthase, γ-terpinene synthase, terpinolene synthase, amorphadiene synthase, α-farnesene synthase, β-farnesene synthase, farnesol synthase, nerolidol synthase, patchouliol synthase, nootkatone synthase, and abietadiene synthase.

11. The genetically modified yeast cell of claim 1, further comprising one or more heterologous nucleotide sequences encoding one or more flocculation proteins.

12. The genetically modified yeast cell of claim 11, wherein the one or more flocculation proteins are selected from the group consisting of Flo1p, Flo5p, Flo8p, Flo9p, Flo10p, and Flo11p.

13. The genetically modified yeast cell of claim 1 that is haploid.

14. The genetically modified yeast cell of claim 1 that is diploid.

15. The genetically modified diploid yeast cell of claim 14 that is heterozygous.

16. The genetically modified diploid yeast cell of claim 15 that is homozygous other than for its mating type allele.

17. The genetically modified yeast cell of claim 1 that is sporulation impaired.

18. The genetically modified yeast cell of claim 17 that is sporulation impaired by virtue of having a functional disruption in a sporulation gene selected from the group consisting of IME1, IME2, NDT80, SPO11, SPO20, AMA1, HOP2, and SPO21.

19. The genetically modified yeast cell of claim 1 that is endogenous mating impaired.

20. The genetically modified yeast cell of claim 19 that is endogenous mating impaired by virtue of having a functional disruption in a pheromone response gene selected from the group consisting of STE5, STE4, STE18, STE12, STE7, and STE11.

21. The genetically modified yeast cell of any one of claims 1-20 that is a Saccharomyces cerevisiae cell.

22. The genetically modified yeast cell of claim 21, wherein the Saccharomyces cerevisiae cell is of the PE-2 strain.

23. A MATα/a ste5/ste5 ime1/ime1 yeast cell that comprises:
(a) one or more heterologous nucleotide sequences encoding one or more enzymes of the MEV pathway; and
(b) one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17.

24. The genetically modified yeast cell of claim 23, wherein the heterologous nucleotide sequence encodes an enzyme that can convert 3-hydroxy-3-methylglutaryl-CoA (HMG-CoA) into mevalonate.

25. The genetically modified yeast cell of claim 23, wherein the heterologous nucleotide sequence encodes an enzyme that can convert mevalonate into mevalonate 5-phosphate.

26. The genetically modified yeast cell of claim 23, further comprising one or more heterologous nucleotide sequences encoding one or more flocculation proteins selected from the group consisting of Flo1p, Flo5p, Flo8p, Flo9p, Flo10p, and Flo11p.

27. The genetically modified yeast cell of any one of claims 23-26 that is a Saccharomyces cerevisiae cell.

28. The genetically modified yeast cell of claim 27, wherein the Saccharomyces cerevisiae cell is of the PE-2 strain.

29. A method for producing an isoprenoid compound comprising:
(a) obtaining a plurality of genetically modified yeast cells that are capable of making said isoprenoid compound and comprising:
(i) one or more heterologous nucleotide sequences encoding one or more enzymes of the MEV pathway; and
(ii) one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17;
(b) culturing said genetically modified yeast cells in a medium with a carbon source under conditions suitable for making said isoprenoid compound; and
(c) recovering said isoprenoid compound from the medium.

30. The method of claim 29, wherein the isoprenoid compound is produced in an amount greater than about 10 grams per liter of medium.

31. The method of claim 29, wherein the isoprenoid compound is produced in an amount greater than about 50 mg per gram of dry cell weight.

32. The method of claim 29, wherein the amount of isoprenoid compound is produced in less than about 72 hours.

33. The method of claim 29, wherein the amount of isoprenoid compound is produced in less than about 48 hours.

34. The method of claim 29, wherein the amount of isoprenoid compound is produced in less than about 24 hours.

35. The method of claim 29, wherein the isoprenoid is a C₅-C₂₀ isoprenoid.

36. The method of claim 35, wherein the isoprenoid is selected from the group consisting of abietadiene, amorphadiene, carene, α-farnesene, β-farnesene, farnesol, geraniol, geranylgeraniol, isoprene, linalool, limonene, myrcene, nerolidol, ocimene, patchoulol, β-pinene, sabinene, γ-terpinene, terpinolene, and valencene.

37. A method for detecting in a biological sample the presence or absence of a genetically modified microbial cell comprising one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17, said method comprising:
(a) obtaining a biological sample;
(b) contacting the biological sample with a first compound or agent capable of interacting with a target molecule, wherein the target molecule is either a nucleic acid comprising a nucleotide sequence encoding a polypeptide having an amino acid sequence that is at least 80% identical to an amino acid sequence selected from the group consisting of SEQ ID NOs: 1, 3, 5, 7, 9, 11, 13, 15, and 17, or a polypeptide having an amino acid sequence that is at least 80% identical to an amino acid sequence selected from the group consisting of SEQ ID NOs: 1, 3, 5, 7, 9, 11, 13, 15, and 17; and
(c) detecting said interaction of said first compound or agent with said target molecule,
wherein detection of said interaction of said first compound or agent with said target molecule indicates the presence in the biological sample of a genetically modified microbial cell comprising one or more nucleotide sequences encoding one or more polypeptides having an amino acid sequence that is at least 80% identical to an amino acid sequence selected from the group consisting of SEQ ID NOs: 1, 3, 5, 7, 9, 11, 13, 15, and 17.

38. The method of claim 37, wherein the first compound or agent is a nucleic acid probe that can hybridize to a nucleic acid encoding a polypeptide having an amino acid sequence that is at least 80% identical to an amino acid sequence selected from the group consisting of SEQ ID NOs: 1, 3, 5, 7, 9, 11, 13, 15, and 17.

39. The method of claim 38, wherein the nucleic acid probe comprises more than 50 nucleotides.

40. The method of claim 38, wherein the nucleic acid probe comprises less than 50 nucleotides.

41. The method of claim 38, wherein the nucleic acid probe is physically linked to a detectable substance.

42. The method of claim 37, wherein the first compound or agent is an antibody or an antibody fragment that that can bind a polypeptide having an amino acid sequence that is at least 80% identical to an amino acid sequence selected from the group consisting of SEQ ID NOs: 1, 3, 5, 7, 9, 11, 13, 15, and 17.

43. The method of claim 42, wherein the antibody or antibody fragment is polyclonal.

44. The method of claim 42, wherein the antibody or antibody fragment is monoclonal.

45. The method of claim 42, wherein the antibody fragment is a Fab fragment.

46. The method of claim 42, wherein the antibody or antibody fragment is physically linked to a detectable substance.

47. The method of claim 41 or claim 46, wherein the detectable substance is a fluorescent molecule.

48. The method of claim 41 or claim 46, wherein the detectable substance is a radioactive isotope.
